(19) United States
(12) Patent Application Publication
(10) Pub. No.: US 2026/0153510 A1
(43) Pub. Date: Jun. 4, 2026

(54) BRIGHTFIELD TRIPLEX IMMUNOHISTOCHEMISTRY ASSAY FOR EVALUATING THE COLOCALIZATION OF THE ER, PR, AND KI-67 BIOMARKERS IN CELLS

(71) Applicants: VENTANA MEDICAL SYSTEMS, INC., Tucson, AZ (US); JAPANESE FOUNDATION FOR CANCER RESEARCH, Tokyo (JP)

(72) Inventors: Rie Horii, Tokyo (JP); Hiro Nitta, Tucson, AZ (US); Takayuki Ueno, Tokyo (JP)

(21) Appl. No.: 19/412,492

(22) Filed: Dec. 8, 2025

Related U.S. Application Data
(63) Continuation of application No. PCT/US2024/036846, filed on Jul. 5, 2024.
(60) Provisional application No. 63/525,406, filed on Jul. 7, 2023.

(51) Int. Cl. G01N 33/57515; G01N 1/30; G01N 33/575; G01N 33/68

(52) U.S. Cl. CPC G01N 33/57515; G01N 1/30; G01N 33/57585; G01N 33/5759; G01N 33/6872; G01N 2333/4706; G01N 2333/723; G01N 2474/20

(57) ABSTRACT

The present disclosure is directed to a triplex immunohistochemical assay for detecting the colocalization of the ER, PR, and Ki-67 biomarkers in cells or cell nuclei. It is believed that the brightfield triplex immunohistochemical assay of the present disclosure may serve as a prognostic assay for ER-positive breast cancer, facilitating the identification of disease-free survivors among ER-positive breast cancer patients treated with hormone therapy.

### CROSS REFERENCE TO RELATED APPLICATIONS

The present disclosure claims the benefit of the filing date of International Application No. PCT/US2024/036846 filed on Jul. 5, 2024, which application claims the benefit of the filing date of U.S. Provisional Patent Application No. 63/525,406 filed on Jul. 7, 2023, the disclosure of which is hereby incorporated by reference herein in its entirety.

### BACKGROUND OF THE DISCLOSURE

Breast cancer accounts for about 23% of all cancers worldwide and is responsible for hundreds of thousands of deaths each year. Breast cancers vary in their response to different treatments, and it is important to select an appropriate treatment regimen for each patient. Receptor status is a common classification system that is used to select treatments for a patient with breast cancer. Breast tumors may have (be positive for) or lack (be negative for) estrogen receptor (ER) protein, HER2 (also known as ErbB2) protein, and/or progesterone receptor (PR) protein. Breast tumors are also routinely screened for HER2 gene amplification, as another measure of whether the tumor is HER2 positive or negative. Some breast tumors are negative for all three markers (ER, PR, and HER2) and are referred to as “triple negative” tumors.

Estrogen receptor and/or progesterone receptor positive tumors are typically treated with hormone-blocking therapy (such as tamoxifen); while HER2 positive tumors are treated with HER2-targeting therapeutics such as trastuzumab or lapatinib.

Another biomarker, Ki-67, is often combined with the biomarkers ER, PR, and HER2 in an immunohistochemical score “IHC4” to evaluate risk of recurrence (for example see Cuzick et al., J. Clin. Oncol. 29:4273-8, 2011, and Barton et al., Br. J. Cancer 1-6, Apr. 24, 2012). The IHC4 score, however, lacks reproducibility.

There remains a need to improve current molecular screening methods to accurately assess the risk for recurrence and to select appropriate therapies in the clinic.

### BRIEF SUMMARY OF THE DISCLOSURE

The present disclosure is directed to a brightfield triplex immunohistochemical (IHC) assay for detecting the colocalization of the ER, PR, and Ki-67 biomarkers in cells or cell nuclei. It is believed that the brightfield triplex immunohistochemical assay of the present disclosure may serve as a prognostic assay for ER-positive breast cancer, facilitating the identification of disease-free survivors among ER-positive breast cancer patients treated with hormone therapy. It is also believed that the brightfield triplex immunohistochemical assay of the present disclosure may serve as a companion assay to identify ER-positive breast cancer patients who should be treated with a CDK4/6 inhibitor because of their poor prognosis. It is further believed that the disclosed brightfield triplex immunohistochemical assay could serve as a replacement for costly gene expression tests (e.g., Oncotype DX, MammaPrint, Prosigna, Breast Cancer Index).

A first aspect of the present disclosure is an affinity histochemical or affinity cytochemical (e.g., immunohistochemical method) for assessing expression of the PR, ER, and Ki-67 biomarkers in a sample, the method comprising: contacting the sample with a human PR biomarker-specific reagent under conditions that permit specific binding of the PR biomarker-specific reagent to the sample; contacting the sample with a first set of detection reagents that interact with the human PR biomarker-specific reagent to facilitate deposition of a first brightfield detectable moiety on the sample; contacting the sample with a human ER biomarker-specific reagent under conditions that permit specific binding of the ER biomarker-specific reagent to the sample; contacting the sample with a second set of detection reagents that interact with the human ER biomarker-specific reagent to facilitate deposition of a second brightfield detectable moiety on the sample; contacting the sample with a human Ki-67 biomarker-specific reagent under conditions that permit specific binding of the Ki-67 biomarker-specific reagent to the sample; contacting the sample with a third set of detection reagents that interact with the human Ki-67 biomarker-specific reagent to facilitate deposition of a third brightfield detectable moiety on the sample; and identifying cell nuclei within the sample expressing each of the PR, ER, and Ki-67 biomarkers based on a colocalization of signals from each of the deposited first, second, and third brightfield detectable moieties. In some embodiments, the first, second, and third brightfield detectable moieties are each different.

In some embodiments, the human PR biomarker-specific reagent is an anti-PR monoclonal antibody. In some embodiments, the human PR biomarker-specific reagent is Clone 1E2.

In some embodiments, the human ER biomarker-specific reagent is an anti-ER monoclonal antibody. In some embodiments, the human ER biomarker-specific reagent is SP1.

In some embodiments, the human Ki-67 biomarker-specific reagent is an anti-Ki-67 monoclonal antibody. In some embodiments, the human Ki-67 biomarker-specific reagent is Clone 30-9.

In some embodiments, the first set of detection reagents include: (i) a first secondary antibody specific to the human PR biomarker-specific reagent; and (ii) a conjugate including the first brightfield detectable moiety. In some embodiments, the first secondary antibody specific to the human PR biomarker-specific reagent includes a first enzyme.

In some embodiments, the second set of detection reagents include: (i) a second secondary antibody specific to the human ER biomarker-specific reagent; and (ii) a conjugate including the second brightfield detectable moiety. In some embodiments, the second secondary antibody specific to the human ER biomarker-specific reagent includes a first enzyme.

In some embodiments, the third set of detection reagents include: (i) a third secondary antibody specific to the human Ki-67 biomarker-specific reagent; and (ii) a conjugate including the third brightfield detectable moiety. In some embodiments, the third secondary antibody specific to the human Ki-67 biomarker-specific reagent includes a first enzyme.

In some embodiments, the first, second, and third brightfield detectable moieties are selected from the group consisting of TAMRA, Dabsyl, Dabcyl, Cy3, CyB, Cy3.5, Cy5, Cy5.5, Cy7, rhodamine 800 and fluorescein.

In some embodiments, the sample is a breast tissue sample. In some embodiments, the breast tissue sample is from a subject diagnosed with breast cancer. In some embodiments, the breast cancer is Luminal A breast cancer.

In some embodiments, an inactivation composition is applied to the sample prior to the contacting the sample with the human ER biomarker-specific reagent. In some embodiments, an inactivation composition is applied to the sample prior to the contacting the sample with the human Ki-67 biomarker-specific reagent.

A second aspect of the present disclosure is a method of selecting a patient with a breast tumor to receive a hormone therapy, the method comprising: affinity histochemically staining a sample derived from the breast tumor with a human PR biomarker-specific reagent under conditions that permit specific binding of the PR biomarker-specific reagent to the sample; affinity histochemically staining the sample derived from the breast tumor with a human ER biomarker-specific reagent under conditions that permit specific binding of the ER biomarker-specific reagent to the sample; affinity histochemically staining the sample derived from the breast tumor with a human Ki-67 biomarker-specific reagent under conditions that permit specific binding of the Ki-67 biomarker-specific reagent to the sample; determining a number of proliferating tumor nuclei (i.e., those nuclei that are Ki-67+) that are both ER+ and PR+ within the histochemically stained sample; wherein the patient is selected to receive the hormone therapy if a ratio of the determined number of proliferating tumor nuclei that are both ER+ and PR+ to a total number of ER+ proliferating tumor nuclei is greater or equal to a predetermined cutoff value. In some embodiments, the histochemical staining of the PR, ER, and Ki-67 biomarkers may be conducted in any order. In other embodiments, histochemical staining of the PR, ER, and Ki-67 biomarkers is conducted in the order set forth above.

In some embodiments, the predetermined cutoff value is between about 0.3 to about 0.7. In some embodiments, the predetermined cutoff value is between about 0.4 to about 0.6. In some embodiments, the predetermined cutoff value is between about 0.45 to about 0.55. In some embodiments, the predetermined cutoff value is about 0.48. In some embodiments, the predetermined cutoff value is about 0.49. In some embodiments, the predetermined cutoff value is about 0.5. In some embodiments, the predetermined cutoff value is about 0.51. In some embodiments, the predetermined cutoff value is about 0.52.

In some embodiments, the hormone therapy is a selective estrogen receptor modulator. In some embodiments, the hormone therapy is a selective estrogen receptor degrader. In some embodiments, the hormone therapy is an aromatase inhibitor. In some embodiments, the patient was previously diagnosed with Luminal A breast cancer. In some embodiments, rein the patient was previously treated with endocrine therapy. In some embodiments, the patient was previously treated with adjuvant chemotherapy.

In some embodiments, the affinity histochemical staining of the sample with the human PR biomarker-specific reagent comprises (i) contacting the sample with a human PR biomarker-specific reagent under conditions that permit specific binding of the PR biomarker-specific reagent to the sample; and (ii) contacting the sample with a first set of detection reagents that interact with the human PR biomarker-specific reagent to facilitate deposition of a first brightfield detectable moiety on the sample. In some embodiments, the affinity histochemical staining of the sample with the human ER biomarker-specific reagent comprises (i) contacting the sample with a human ER biomarker-specific reagent under conditions that permit specific binding of the ER biomarker-specific reagent to the sample; and (ii) contacting the sample with a second set of detection reagents that interact with the human ER biomarker-specific reagent to facilitate deposition of a second brightfield detectable moiety on the sample. In some embodiments, the affinity histochemical staining of the sample with the human Ki-67 biomarker-specific reagent comprises (i) contacting the sample with a human Ki-67 biomarker-specific reagent under conditions that permit specific binding of the Ki-67 biomarker-specific reagent to the sample; and (ii) contacting the sample with a third set of detection reagents that interact with the human Ki-67 biomarker-specific reagent to facilitate deposition of a third brightfield detectable moiety on the sample.

In some embodiments, the determining of the number of proliferating tumor nuclei that are both ER+ and PR+ within the affinity histochemically stained sample comprises identifying cell nuclei within the sample expressing each of the PR, ER, and Ki-67 biomarkers, such as based on a colocalization of signals from each of the deposited first, second, and third brightfield detectable moieties. In some embodiments, the first, second, and third brightfield detectable moieties are selected from the group consisting of TAMRA, Dabsyl, Dabcyl, Cy3, CyB, Cy3.5, Cy5, Cy5.5, Cy7, rhodamine 800 and fluorescein. In some embodiments, the first, second, and third brightfield detectable moieties are each different.

A third aspect of the present disclosure is a method of selecting a patient with a breast tumor to receive a hormone therapy, the method comprising: affinity histochemically staining a sample derived from the breast tumor with a human PR biomarker-specific reagent under conditions that permit specific binding of the PR biomarker-specific reagent to the sample; affinity histochemically staining the sample derived from the breast tumor with a human ER biomarker-specific reagent under conditions that permit specific binding of the ER biomarker-specific reagent to the sample; affinity histochemically staining the sample derived from the breast tumor with a human Ki-67 biomarker-specific reagent under conditions that permit specific binding of the Ki-67 biomarker-specific reagent to the sample; calculating a ratio of a number of cells or cell nuclei that stain positive for all three of ER, Ki-67, and PR to the total number of cells that are both (i) ER+, Ki-67+, and PR+, and (ii) ER+, Ki-67+, and PR−; wherein the patient is selected to receive the hormone therapy if the calculated ratio is greater than or equal to a predetermined cutoff value. In some embodiments, the histochemical staining of the PR, ER, and Ki-67 biomarkers may be conducted in any order. In other embodiments, histochemical staining of the PR, ER, and Ki-67 biomarkers is conducted in the order set forth above.

In some embodiments, the predetermined cutoff value is between about 0.4 to about 0.6. In some embodiments, the predetermined cutoff value is between about 0.45 to about 0.55. In some embodiments, the predetermined cutoff value is about 0.5. In some embodiments, the ratio is calculated using the formula:

$$[ E {R}^{+} , Ki - {67}^{+} , {PR}^{+} ] / ( [ E {R}^{+} , Ki - {67}^{+} , {PR}^{+} ] + [ E {R}^{+} , Ki - {67}^{+} , {PR}^{-} ] ) .$$

In some embodiments, the affinity histochemical staining of the sample with the human PR biomarker-specific reagent comprises (i) contacting the sample with a human PR biomarker-specific reagent under conditions that permit specific binding of the PR biomarker-specific reagent to the sample; and (ii) contacting the sample with a first set of detection reagents that interact with the human PR biomarker-specific reagent to facilitate deposition of a first brightfield detectable moiety on the sample. In some embodiments, the affinity histochemical staining of the sample with the human ER biomarker-specific reagent comprises (i) contacting the sample with a human ER biomarker-specific reagent under conditions that permit specific binding of the ER biomarker-specific reagent to the sample; and (ii) contacting the sample with a second set of detection reagents that interact with the human ER biomarker-specific reagent to facilitate deposition of a second brightfield detectable moiety on the sample. In some embodiments, the affinity histochemical staining of the sample with the human Ki-67 biomarker-specific reagent comprises (i) contacting the sample with a human Ki-67 biomarker-specific reagent under conditions that permit specific binding of the Ki-67 biomarker-specific reagent to the sample; and (ii) contacting the sample with a third set of detection reagents that interact with the human Ki-67 biomarker-specific reagent to facilitate deposition of a third brightfield detectable moiety on the sample. In some embodiments, the first, second, and third brightfield detectable moieties are selected from the group consisting of TAMRA, Dabsyl, Dabcyl, Cy3, CyB, Cy3.5, Cy5, Cy5.5, Cy7, rhodamine 800 and fluorescein.

A fourth aspect of the present disclosure is a method of selecting a patient with a breast tumor to receive treatment with a cyclin-dependent kinase 4 and 6 inhibitor, the method comprising: affinity histochemically staining a sample derived from the breast tumor with a human PR biomarker-specific reagent under conditions that permit specific binding of the PR biomarker-specific reagent to the sample; affinity histochemically staining the sample derived from the breast tumor with a human ER biomarker-specific reagent under conditions that permit specific binding of the ER biomarker-specific reagent to the sample; affinity histochemically staining the sample derived from the breast tumor with a human Ki-67 biomarker-specific reagent under conditions that permit specific binding of the Ki-67 biomarker-specific reagent to the sample; calculating a ratio of a number of cells or cell nuclei that stain positive for all three of ER, Ki-67, and PR to the total number of cells that are (i) ER+, Ki-67+, and PR+, and (ii) ER+, Ki-67+, and PR−; wherein the patient is selected to receive the hormone therapy if the calculated ratio is less than a predetermined cutoff value. In some embodiments, the histochemical staining of the PR, ER, and Ki-67 biomarkers may be conducted in any order. In other embodiments, histochemical staining of the PR, ER, and Ki-67 biomarkers is conducted in the order set forth above.

In some embodiments, the predetermined cutoff value is between about 0.4 to about 0.6. In some embodiments, the predetermined cutoff value is between about 0.45 to about 0.55. In some embodiments, the predetermined cutoff value is about 0.5. In some embodiments, the ratio is calculated using the formula:

$$[ E {R}^{+} , Ki - {67}^{+} , {PR}^{+} ] / ( [ E {R}^{+} , Ki - {67}^{+} , {PR}^{+} ] + [ E {R}^{+} , Ki - {67}^{+} , {PR}^{-} ] ) .$$

In some embodiments, the affinity histochemical staining of the sample with the human PR biomarker-specific reagent comprises (i) contacting the sample with a human PR biomarker-specific reagent under conditions that permit specific binding of the PR biomarker-specific reagent to the sample; and (ii) contacting the sample with a first set of detection reagents that interact with the human PR biomarker-specific reagent to facilitate deposition of a first brightfield detectable moiety on the sample. In some embodiments, the affinity histochemical staining of the sample with the human ER biomarker-specific reagent comprises (i) contacting the sample with a human ER biomarker-specific reagent under conditions that permit specific binding of the ER biomarker-specific reagent to the sample; and (ii) contacting the sample with a second set of detection reagents that interact with the human ER biomarker-specific reagent to facilitate deposition of a second brightfield detectable moiety on the sample. In some embodiments, the affinity histochemical staining of the sample with the human Ki-67 biomarker-specific reagent comprises (i) contacting the sample with a human Ki-67 biomarker-specific reagent under conditions that permit specific binding of the Ki-67 biomarker-specific reagent to the sample; and (ii) contacting the sample with a third set of detection reagents that interact with the human Ki-67 biomarker-specific reagent to facilitate deposition of a third brightfield detectable moiety on the sample. In some embodiments, the first, second, and third brightfield detectable moieties are selected from the group consisting of TAMRA, Dabsyl, Dabcyl, Cy3, CyB, Cy3.5, Cy5, Cy5.5, Cy7, rhodamine 800 and fluorescein.

A fifth aspect of the present disclosure is a method of classifying a patient with breast cancer as either ER+, Ki-67+, PR+ dominant or ER+, Ki-67+ dominant, the method comprising: affinity histochemically staining a sample derived from the patient with a human PR biomarker-specific reagent under conditions that permit specific binding of the PR biomarker-specific reagent to the sample; affinity histochemically staining the sample derived from the patient with a human ER biomarker-specific reagent under conditions that permit specific binding of the ER biomarker-specific reagent to the sample; affinity histochemically staining the sample derived from the patient with a human Ki-67 biomarker-specific reagent under conditions that permit specific binding of the Ki-67 biomarker-specific reagent to the sample; calculating a ratio of a number of cells or cell nuclei that stain positive for all three of ER, Ki-67, and PR to the total number of cells that are (i) ER+, Ki-67+, and PR+, and (ii) ER+, Ki-67+, and PR−; wherein the patient is classified as ER+, Ki-67+, PR+ dominant if the calculated ratio is greater than or equal to a predetermined threshold; or wherein the patient is classified as ER+, Ki-67+ dominant if the calculated ratio is less than the predetermined threshold. In some embodiments, the affinity histochemical staining of the PR, ER, and Ki-67 biomarkers may be conducted in any order. In other embodiments, histochemical staining of the PR, ER, and Ki-67 biomarkers is conducted in the order set forth above.

In some embodiments, the predetermined cutoff value is between about 0.4 to about 0.6. In some embodiments, the predetermined cutoff value is between about 0.45 to about 0.55. In some embodiments, the predetermined cutoff value is about 0.5.

In some embodiments, if the patient is classified as ER+, Ki-67+, PR+ dominant, then the patient is selected to receive hormone therapy. In some embodiments, if the patient is classified as ER+, Ki-67+ dominant, then the patient is selected to receive a cyclin-dependent kinase 4 and 6 inhibitor.

In some embodiments, the affinity histochemical staining of the sample with the human PR biomarker-specific reagent comprises (i) contacting the sample with a human PR biomarker-specific reagent under conditions that permit specific binding of the PR biomarker-specific reagent to the sample; and (ii) contacting the sample with a first set of detection reagents that interact with the human PR biomarker-specific reagent to facilitate deposition of a first brightfield detectable moiety on the sample. In some embodiments, the affinity histochemical staining of the sample with the human ER biomarker-specific reagent comprises (i) contacting the sample with a human ER biomarker-specific reagent under conditions that permit specific binding of the ER biomarker-specific reagent to the sample; and (ii) contacting the sample with a second set of detection reagents that interact with the human ER biomarker-specific reagent to facilitate deposition of a second brightfield detectable moiety on the sample. In some embodiments, the affinity histochemical staining of the sample with the human Ki-67 biomarker-specific reagent comprises (i) contacting the sample with a human Ki-67 biomarker-specific reagent under conditions that permit specific binding of the Ki-67 biomarker-specific reagent to the sample; and (ii) contacting the sample with a third set of detection reagents that interact with the human Ki-67 biomarker-specific reagent to facilitate deposition of a third brightfield detectable moiety on the sample. In some embodiments, the first, second, and third brightfield detectable moieties are selected from the group consisting of TAMRA, Dabsyl, Dabcyl, Cy3, CyB, Cy3.5, Cy5, Cy5.5, Cy7, rhodamine 800 and fluorescein.

A sixth aspect of the present disclosure is a method of classifying a patient with breast cancer as a likely responder or a likely non-responder to hormone treatment, the method comprising: affinity histochemically staining a sample derived from the patient with a human PR biomarker-specific reagent under conditions that permit specific binding of the PR biomarker-specific reagent to the sample; affinity histochemically staining the sample derived from the patient with a human ER biomarker-specific reagent under conditions that permit specific binding of the ER biomarker-specific reagent to the sample; affinity histochemically staining the sample derived from the patient with a human Ki-67 biomarker-specific reagent under conditions that permit specific binding of the Ki-67 biomarker-specific reagent to the sample; calculating a ratio of a number of cells or cell nuclei that stain positive for all three of ER, Ki-67, and PR to the total number of cells that are (i) ER+, Ki-67+, and PR+, and (ii) ER+, Ki-67+, and PR−; wherein the patient is classified as likely responder if the calculated ratio is greater than or equal to a predetermined threshold; or wherein the patient is classified as a likely non-responder if the calculated ratio is less than the predetermined threshold. In some embodiments, the histochemical staining of the PR, ER, and Ki-67 biomarkers may be conducted in any order. In other embodiments, histochemical staining of the PR, ER, and Ki-67 biomarkers is conducted in the order set forth above.

### BRIEF DESCRIPTION OF THE FIGURES

The patent or application file contains at least one drawing executed in color. Copies of this patent or patent application publication with color drawings will be provided to the Office upon request and the payment of the necessary fee.

FIG. 1 illustrates a method of assessing the ER, Ki-67, and PR biomarkers in a sample using a triplex immunohistochemical assay in accordance with one embodiment of the present disclosure.

FIG. 2 illustrates a method of assessing the ER, Ki-67, and PR biomarkers in a sample using a triplex immunohistochemical assay in accordance with one embodiment of the present disclosure.

FIG. 3 illustrates a method of assessing the ER, Ki-67, and PR biomarkers in a sample using a triplex immunohistochemical assay in accordance with one embodiment of the present disclosure, and further illustrates the selection of different brightfield dyes for staining each of the different biomarkers.

FIGS. 4A-4C illustrate simplex immunohistochemical assays, where a different biomarker is stained with a different brightfield dye. FIG. 4A shows the staining of cells purple; FIG. 4B shows the staining of cells blue/cyan; and FIG. 4C shows the staining of cells yellow.

FIGS. 5A-5C illustrate the colocalization of three different signals from three different brightfield dyes in cells or cell nuclei, where “PR” is purple, “ER” is blue/cyan; and “Ki-67” is yellow.

FIG. 6A illustrates the colocalization of three different signals in a sample stained with three different brightfield dyes, such as colocalized signals from purple, blue/cyan, and yellow stains.

FIG. 6B illustrates the colocalization of two different signals in a sample stained with three different brightfield dyes.

FIG. 7 depicts the disease-free survival of ER+, Ki-67+, PR+ dominant cases and an ER+, Ki-67+ dominant cases.

FIG. 8 shows breast cancer-specific survival of ER+, Ki-67+, PR+ dominant cases and an ER+, Ki-67+ dominant cases.

FIG. 9 illustrates the reclassification of Luminal A breast cancer into an ER+, Ki-67+, PR+ dominant subtype and an ER+, Ki-67+ dominant subtype.

FIG. 10 illustrates a method of treating a subject having breast cancer after classifying a sample derived from the subject as Ki-67+, Ki-67+, PR+ dominant or Ki-67+, PR+ dominant in accordance with one embodiment of the present disclosure.

### DETAILED DESCRIPTION

It should also be understood that, unless clearly indicated to the contrary, in any methods claimed herein that include more than one step or act, the order of the steps or acts of the method is not necessarily limited to the order in which the steps or acts of the method are recited.

As used herein, the singular terms “a,” “an,” and “the” include plural referents unless context clearly indicates otherwise. Similarly, the word “or” is intended to include “and” unless the context clearly indicates otherwise. The term “includes” is defined inclusively, such that “includes A or B” means including A, B, or A and B.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one of a number or list of elements, and, optionally, additional unlisted items. Only terms clearly indicated to the contrary, such as “only one of or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of” or “exactly one of.” “Consisting essentially of,” when used in the claims, shall have its ordinary meaning as used in the field of patent law.

The terms “comprising,” “including,” “having,” and the like are used interchangeably and have the same meaning. Similarly, “comprises,” “includes,” “has,” and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a device having components a, b, and c” means that the device includes at least components a, b, and c. Similarly, the phrase: “a method involving steps a, b, and c” means that the method includes at least steps a, b, and c. Moreover, while the steps and processes may be outlined herein in a particular order, the skilled artisan will recognize that the ordering steps and processes may vary.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, the term “administering” refers to the physical introduction of a composition comprising a therapeutic agent to a subject, using any of the various methods and delivery systems known to those skilled in the art. Routes of administration for the formulations disclosed herein include intravenous, intramuscular, subcutaneous, intraperitoneal, spinal, or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In some embodiments, the formulation is administered via a non-parenteral route, in some embodiments, orally. Other non-parenteral routes include a topical, epidermal, or mucosal route of administration, for example, intranasally, vaginally, rectally, sublingually, or topically. Administering can also be performed, for example, once, a plurality of times, and/or over one or more extended periods.

As used herein, the term “antibody” refers to and encompasses various antibody structures, including but not limited to monoclonal antibodies, polyclonal antibodies, multispecific antibodies (e.g., bispecific antibodies), and antibody fragments so long as they exhibit the desired antigen-binding activity.

As used herein, the term “antibody fragment” refers to a molecule other than an intact antibody that includes a portion of an intact antibody that binds the antigen to which the intact antibody binds. Examples of antibody fragments include but are not limited to Fv, Fab, Fab,′ Fab′-SH, F(ab′)2; diabodies; linear antibodies; single-chain antibody molecules (e.g., scFv); and multispecific antibodies formed from antibody fragments.

As use herein, the terms “binds,” “specific binding,” “specifically binds to” or is “specific for” refers to measurable and reproducible interactions such as binding between a target and a specific binding agent, which is determinative of the presence of the target in the presence of a heterogeneous population of molecules including biological molecules. For example, a binding entity that specifically binds to a target may be an antibody that binds the target with greater affinity, avidity, more readily, and/or with greater duration than it binds to other targets. In one embodiment, the extent of binding of an antibody to an unrelated target is less than about 10% of the binding of the antibody to the target as measured, e.g., by a radioimmunoassay (RIA). In certain embodiments, an antibody that specifically binds to a target has a dissociation constant (Kd) of ≤1 μM, ≤100 nM, ≤10 nM, ≤1 nM, or ≤0.1 nM. In certain embodiments, an antibody specifically binds to an epitope on a protein that is conserved among the protein from different species. In another embodiment, specific binding can include, but does not require exclusive binding.

As used herein, the term “biomarker” refers to any molecule or group of molecules found in a biological sample that can be used to characterize the biological sample or a subject from which the biological sample is obtained. For example, a biomarker may be a molecule or group of molecules whose presence, absence, or relative abundance is characteristic of a particular cell or tissue type or state; or characteristic of a particular pathological condition or state; or indicative of the severity of a pathological condition, the likelihood of progression or regression of the pathological condition, and/or the likelihood that the pathological condition will respond to a particular treatment. As another example, the biomarker may be a cell type or a microorganism (such as a bacterium, mycobacterium, fungus, virus, and the like), or a substituent molecule or group of molecules thereof.

As used herein, the phrase “biomarker specific reagent” refers to a specific detection reagent that is capable of specifically binding directly to one or more biomarkers in the cellular sample, such as a primary antibody.

As used herein, the term “cancer” refers a broad group of various diseases characterized by the uncontrolled growth of abnormal cells in the body. Unregulated cell division and growth results in the formation of malignant tumors that invade neighboring tissues and may also metastasize to distant parts of the body through the lymphatic system or bloodstream. The term “cancer” is generally used interchangeably with “tumor” herein (unless a tumor is specifically referred to as a “benign” tumor, which is an abnormal mass of cells that lacks the ability to invade neighboring tissue or metastasize), and encompasses malignant solid tumors (e.g., carcinomas, sarcomas) and malignant growths in which there may be no detectable solid tumor mass (e.g., certain hematologic malignancies). Non-limiting examples of cancers include but are not limited to, carcinoma, lymphoma, blastoma, sarcoma, and leukemia or lymphoid malignancies. More particular examples of such cancers include, but not limited to, squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer and gastrointestinal stromal cancer, pancreatic cancer, glioblastoma, cervical cancer, ovarian cancer, liver cancer, bladder cancer, cancer of the urinary tract, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, melanoma, superficial spreading melanoma, lentigo maligna melanoma, acral lentiginous melanomas, nodular melanomas, multiple myeloma and B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); hairy cell leukemia; chronic myeloblastic leukemia; and post-transplant lymphoproliferative disorder (PTLD), as well as abnormal vascular proliferation associated with phacomatoses, edema (such as that associated with brain tumors), Meigs' syndrome, brain, as well as head and neck cancer, and associated metastases. In certain embodiments, cancers that are amenable to treatment by the antibodies of the present disclosure include breast cancer, colorectal cancer, rectal cancer, non-small cell lung cancer, glioblastoma, non-Hodgkin's lymphoma (NHL), renal cell cancer, prostate cancer, liver cancer, pancreatic cancer, soft-tissue sarcoma, Kaposi's sarcoma, carcinoid carcinoma, head and neck cancer, ovarian cancer, mesothelioma, and multiple myeloma. In some embodiments, the cancer is selected from: small cell lung cancer, glioblastoma, neuroblastomas, melanoma, breast carcinoma, gastric cancer, colorectal cancer (CRC), and hepatocellular carcinoma. Yet in some embodiments, the cancer is selected from: non-small cell lung cancer, colorectal cancer, glioblastoma and breast carcinoma, including metastatic forms of those cancers. In specific embodiments, the cancer is melanoma or lung cancer, suitably metastatic melanoma, or metastatic lung cancer.

As used herein, the terms “chromogen,” “chromogenic compound,” “detectable mostly” or “dye” and the like refer to a substance that can be converted into a colored compound under specific conditions, e.g., when acted upon by an enzyme or under specific chemical/reaction conditions. Examples of enzyme-substrate combinations include: (i) Horseradish peroxidase (HRP) with hydrogen peroxidase as a substrate, where the hydrogen peroxidase oxidizes a dye precursor [e.g., orthophenylene diamine (OPD) or 3,3′,5,5′-tetramethyl benzidine hydrochloride (TMB)]; (ii) alkaline phosphatase (AP) with para-Nitrophenyl phosphate as chromogenic substrate; and (iii) P-D-galactosidase (β-D-Gal) with a chromogenic substrate (e.g., p-nitrophenyl-β-D-galactosidase) or fluorogenic substrate (e.g., 4-methylumbelliferyl-β-D-galactosidase). Numerous other enzyme-substrate combinations are available to those skilled in the art. For a general review of these, see U.S. Pat. Nos. 4,275,149 and 4,318,980.

As used herein, the terms “colocalize” or “colocalization” refer to the occurrence at the same or substantially the same place, such as a stain occurring or being located at the same or substantially the same place.

As used herein, the term “detection reagent” refers to any reagent used to deposit a detectable moiety in proximity to a biomarker-specific reagent bound to a biomarker in a cellular sample to thereby stain the sample. Non-limiting examples include secondary detection reagents (such as secondary antibodies capable of binding to a primary antibody, anything that specifically binds biotin or avidin), tertiary detection reagents (such as tertiary antibodies capable of binding to secondary antibodies), enzymes directly or indirectly associated with the specific binding agent, chemicals reactive with such enzymes to effect deposition of a fluorescent or chromogenic stain, wash reagents used between staining steps, and the like.

As used herein, the term “estrogen receptor” or “ER” refers to a member of the nuclear hormone family of intracellular receptors is activated by 17β-estradiol. Estrogen receptors are overexpressed in around 70% of breast cancer cases, referred to as “ER positive” (ER+). The ESR1 gene encodes an estrogen receptor and ligand-activated transcription factor. The canonical protein contains an N-terminal ligand-independent transactivation domain, a central DNA binding domain, a hinge domain, and a C-terminal ligand-dependent transactivation domain. The protein localizes to the nucleus where it may form either a homodimer or a heterodimer with estrogen receptor 2. The protein encoded by this gene regulates the transcription of many estrogen-inducible genes that play a role in growth, metabolism, sexual development, gestation, and other reproductive functions and is expressed in many non-reproductive tissues. The receptor encoded by the ESR1 gene plays a key role in breast cancer, endometrial cancer, and osteoporosis.

As used herein, the term “formalin-fixed paraffin embedded (FFPE) tissue section” refers to a piece of tissue, e.g., a biopsy that has been obtained from a subject, fixed in formaldehyde (e.g., 3%-5% formaldehyde in phosphate buffered saline) or Bouin solution, embedded in wax, cut into thin sections, and then mounted on a planar surface, e.g., a microscope slide.

As used herein, the term “monoclonal antibody” (“mAb”) refers to a non-naturally occurring preparation of antibody molecules of single molecular composition, i.e., antibody molecules whose primary sequences are essentially identical, and which exhibits a single binding specificity and affinity for a particular epitope. A mAb is an example of an isolated antibody. MAbs may be produced by hybridoma, recombinant, transgenic, or other techniques known to those skilled in the art.

As used herein, the term “immunohistochemistry” refers to a method of determining the presence or distribution of an antigen in a sample by detecting interaction of the antigen with a specific binding agent, such as an antibody. A sample is contacted with an antibody under conditions permitting antibody-antigen binding. Antibody-antigen binding can be detected by means of a detectable label conjugated to the antibody (direct detection) or by means of a detectable label conjugated to a secondary antibody, which binds specifically to the primary antibody (indirect detection).

As used herein, the term “Ki-67” refers to a nuclear protein associated with cellular proliferation and ribosomal RNA transcription. Inactivation of antigen Ki-67 leads to inhibition of ribosomal RNA synthesis. Ki-67 is used, for example, as a marker of proliferation. The MIK67 gene facilitates protein C-terminus binding activity; and is involved in the regulation of chromosome segregation and regulation of mitotic nuclear division.

As used herein, the terms “primary antibody” and “secondary antibody” refer to different antibodies, where a primary antibody is a polyclonal or monoclonal antibody from one species (rabbit, mouse, goat, donkey, etc.) that specifically recognizes an antigen (e.g., a biomarker) in a sample (e.g., a human biological sample) under study, and a secondary antibody is an antibody (usually polyclonal) from a different species that specifically recognizes the primary antibody, e.g., in its Fc region.

As used herein, the term “progesterone receptor” or “PR” refers to an intracellular steroid receptor that specifically binds progesterone. Progesterone receptors are overexpressed in some breast cancer cases, referred to as “PR positive” (PR+). The PGR gene encodes a member of the steroid receptor superfamily. The encoded protein mediates the physiological effects of progesterone, which plays a central role in reproductive events associated with the establishment and maintenance of pregnancy. This gene uses two distinct promotors and translation start sites in the first exon to produce several transcript variants, both protein coding and non-protein coding. Two of the isoforms (A and B) are identical except for an additional 165 amino acids found in the N-terminus of isoform B and mediate their own response genes and physiologic effects with little overlap.

As used herein, the term “sample” shall refer to any material obtained from a subject capable of being tested for the presence or absence of a biomarker, e.g., a tissue sample or a cytology sample.

As used herein, the term “slide” refers to any substrate (e.g., substrates made, in whole or in part, glass, quartz, plastic, silicon, etc.) of any suitable dimensions on which a cellular sample is placed for analysis, and for example, a “microscope slide” such as a standard 3 inch by 1 inch microscope slide or a standard 75 mm by 25 mm microscope slide.

When used as a noun, the term “stain” shall refer to any substance that can be used to visualize specific molecules or structures in a cellular sample for microscopic analysis, including brightfield microscopy, fluorescent microscopy, electron microscopy, and the like. When used as a verb, the term “stain” shall refer to any process that results in deposition of a stain on a cellular sample.

As used herein, the term “subject” or “individual” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

As used herein, the term “tumor” refers to all neoplastic cell growth and proliferation, whether malignant or benign, and all pre-cancerous and cancerous cells and tissues. In some embodiments, the tumor is a malignant cancerous tumor (i.e., cancer). In some embodiments, the tumor is a solid tumor or a non-solid or soft tissue tumor. Examples of soft tissue tumors include leukemia (e.g., chronic myelogenous leukemia, acute myelogenous leukemia, adult acute lymphoblastic leukemia, acute myelogenous leukemia, mature B-cell acute lymphoblastic leukemia, chronic lymphocytic leukemia, prolymphocytic leukemia, or hairy cell leukemia) or lymphoma (e.g., non-Hodgkin's lymphoma, cutaneous T-cell lymphoma, or Hodgkin's disease). A solid tumor includes any cancer of body tissues other than blood, bone marrow, or the lymphatic system. Solid tumors can be further divided into those of epithelial cell origin and those of non-epithelial cell origin. Examples of epithelial cell solid tumors include tumors of the gastrointestinal tract, colon, colorectal (e.g., basaloid colorectal carcinoma), breast, prostate, lung, kidney, liver, pancreas, ovary (e.g., endometrioid ovarian carcinoma), head and neck, oral cavity, stomach, duodenum, small intestine, large intestine, anus, gall bladder, labium, nasopharynx, skin, uterus, male genital organ, urinary organs (e.g., urothelium carcinoma, dysplastic urothelium carcinoma, transitional cell carcinoma), bladder, and skin. Solid tumors of non-epithelial origin include sarcomas, brain tumors, and bone tumors.

### OVERVIEW

The present disclosure is directed to a multiplex immunohistochemical assay for evaluating the expression of the ER, PR, and Ki-67 biomarkers in a sample, such as by detecting the colocalization of the ER, PR, and Ki-67 biomarkers in cells or cell nuclei. It is believed that the multiplex immunohistochemical assay of the present disclosure may serve as a prognostic assay for ER-positive breast cancer, facilitating the identification of disease-free survivors among ER-positive breast cancer patients treated with hormone therapy. Indeed, Applicant has surprisingly discovered that the ER and PR status of proliferating cancer cells (i.e., those cancer cells that are Ki-67 positive) is an effective prognostic factor in ER-positive breast cancer patients.

### Methods of Assessing the Expression of ER, KI-67, and PR Biomarkers

The present disclosure provides for methods of assessing the expression of the ER, Ki-67, and PR biomarkers, such as the colocalized expression of the each of the ER, Ki-67, and PR biomarkers, in cell nuclei.

In some embodiments, the methods employ an affinity histochemical or affinity cytochemical assay so as to stain each of the ER, Ki-67, and PR biomarkers, such as with a different detectable moiety. Affinity histochemical and cytochemical staining techniques typically involve contacting a sample deposited on a slide or other solid support or substrate with a biomarker-specific reagent under conditions sufficient to permit specific binding between the biomarker-specific reagent and the biomarker of interest. Binding of the biomarker-specific reagent to the biomarker facilitates deposition of a detectable moiety on the sample in proximity to locations containing the biomarker. The detectable moiety can be used to locate and/or quantify the biomarker to which the biomarker-specific reagent is directed. Thereby, the presence and/or relative amount of the target in a sample can be detected by detecting the signal produced by the detectable moiety.

In some embodiments, the methods employ a multiplex affinity histochemical or affinity cytochemical assay, e.g., an immunohistochemical assay, where a single tissue sample is stained for the presence of the ER, Ki-67, and PR biomarkers. In some embodiments, the multiplex affinity histochemical or affinity cytochemical assays, e.g., immunohistochemical assays, of the present disclosure employ different brightfield detectable moieties such that each of the ER, Ki-67, and PR biomarkers are stained with a different brightfield detectable moiety. After the tissue sample is stained in the multiplex affinity histochemical or affinity cytochemical assay, e.g., immunohistochemical assay, the stained sample may be evaluated manually or using one or more automated digital pathology techniques. The evaluated stained sample may then be used for risk assessment and/or to selected treatment options. These and other embodiments are described further herein.

### Affinity Histochemical or Affinity Cytochemical Assays

In some embodiments, the expression of the ER, Ki-67, and/or PR biomarkers is assessed using a technique such as immunohistochemistry (IHC). In some embodiments, the sample is a tissue section (including, but not limited to formalin-fixed paraffin embedded (FFPE) tissue sections and fresh frozen tissue sections).

IHC assays involve contacting a sample with a biomarker-specific reagent under conditions that facilitate specific binding between a biomarker (e.g., ER, PR, and/or Ki-67) and a biomarker-specific reagent (also referred to herein as “specific binding entities” or “specific binding agents”) and unbound biomarker-specific reagent is removed from the sample (such as by washing with a wash buffer). If the biomarker-specific reagent is directly conjugated to a detectable moiety (termed a “direct detection IHC assay”), the sample may then be directly analyzed. For instance, the biomarker-specific reagent may be directly conjugated to a brightfield detectable moiety and directly analyzed.

Alternatively, the sample may be contacted with a set of detection reagents that interact with the biomarker-specific reagent to facilitate deposition of a detectable moiety onto or in close proximity the biomarker, thereby generating a detectable signal localized to the biomarker. For instance, the detection reagents may comprise a secondary antibody specific to the biomarker-specific reagent, where the secondary antibody is conjugated to an enzyme. The detection reagents may further comprise a conjugate including a brightfield detectable moiety, where the conjugate is acted upon by the enzyme conjugated to the secondary antibody to deposit the brightfield detectable moiety onto or in proximity to the biomarker.

Typically, one or more inactivation and/or wash steps are performed between application of different reagents to avoid non-specific staining of tissues. Biomarker-labeled samples may optionally be additionally labeled with a contrast agent (such as a hematoxylin stain) to visualize macromolecular structures within the cellular sample. The sample may Aldo be tested for one or more additional biomarkers.

### Samples

Any type of sample compatible with an IHC assay may be used.

In some embodiments, the sample can be a tissue section or a tissue section that has been processed to remove at least part of the tissue section, for example, for further genetic analysis. In some embodiments, the sample can be a cytology sample, either deposited or printed onto a microscope slide. In some embodiments, the cytology sample can be in the form of a cell block that is sectioned, such as a cell block formed from paraffin or other matrix such as gels, aerogels, polymers, proteins that hold the cells in a 3D structure for sectioning. In some embodiments, the cytology sample can be in the form of a cell pellet formed, for example, by sedimentation or centrifugation, such as a pellet in sectionable form for placement onto a microscope slide.

In other embodiments, the sample is a fine needle aspirate or in the form of a core extracted from a tissue sample, either in vivo or ex vivo. In still further embodiments, the sample can be a representative sample, such as described in United States Patent Publication No. 2020/0049599, the disclosure of which is hereby incorporated by reference herein in its entirety.

Samples for use in the methods disclosed herein can be prepared using any method known in the art. The samples can be obtained from a subject for routine screening or from a subject that is suspected of having a disorder or suspected as having a disorder, such as cancer. In some embodiments, the sample is derived from a subject diagnosed as having cancer, e.g., previously diagnosed as having breast cancer, or suspected of having cancer, e.g., suspected of having breast cancer.

In some embodiments, the sample is derived from a tumor of the subject, such as a subject diagnosed as having breast cancer or suspected of having breast cancer. In some embodiments, the sample is derived from a metastatic breast tumor of the subject. In some embodiments, the sample is derived from a patient previously diagnosed with Luminal A breast cancer. In some embodiments, the sample is derived from a patient previously diagnosed with Luminal B breast cancer. In some embodiments, the sample is derived from a subject who has undergone previous treatment for cancer, e.g., breast cancer or Luminal A breast cancer; or is currently undergoing treatment for cancer, e.g., breast cancer or Luminal A breast cancer.

In other embodiments, the samples may not have any abnormalities, diseases, disorders, etc., referred to as “normal” samples. Such normal samples are useful, among other things, as controls for comparison to other samples. For example, it may be useful to test a patient, i.e., a human subject, for cancer by taking tissue samples from multiple locations, and these samples may be used as controls and compared to later samples to determine whether a particular cancer has spread beyond its primary origin.

In some embodiments, the samples have been previously screened in one or more genetic tests. In some embodiments, the samples have been previously determined to be ER positive. In some embodiments, the sample have been previously determined to include proliferating cells, such as by evaluating the expression of one or more proliferation biomarkers.

In some embodiments, the sample is a fixed sample. Fixing a sample preserves cells and tissue constituents in as close to a life-like state as possible and allows them to undergo preparative procedures without significant change. Autolysis and bacterial decomposition processes that begin upon cell death are arrested, and the cellular and tissue constituents of the sample are stabilized so that they withstand the subsequent stages of tissue processing. Fixatives can be classified as cross-linking agents (such as aldehydes, e.g., formaldehyde, paraformaldehyde, and glutaraldehyde, as well as non-aldehyde cross-linking agents), oxidizing agents (e.g., metallic ions and complexes, such as osmium tetroxide and chromic acid), protein-denaturing agents (e.g., acetic acid, methanol, and ethanol), fixatives of unknown mechanism (e.g., mercuric chloride, acetone, and picric acid), combination reagents (e.g., Carnoy's fixative, methacarn, Bouin's fluid, B5 fixative, Rossman's fluid, and Gendre's fluid), microwaves, and miscellaneous fixatives (e.g., excluded volume fixation and vapor fixation). Additives may also be included in the fixative, such as buffers, detergents, tannic acid, phenol, metal salts (such as zinc chloride, zinc sulfate, and lithium salts), and lanthanum. The most commonly used fixative in preparing samples is formaldehyde, generally in the form of a formalin solution (formaldehyde in an aqueous (and typically buffered) solution). In some embodiments, the samples used in the present methods are fixed by a method comprising fixation in a formalin-based fixative. In one example, the fixative is 10% neutral buffered formalin. Notwithstanding these examples, the tissues can be fixed by process using any fixation medium that is compatible with the biomarker-specific reagents and specific detection reagents used.

In some embodiments, the fixed sample is embedded in an embedding medium. An embedding medium is an inert material in which tissues and/or cells are embedded to help preserve them for future analysis. Embedding also enables samples to be sliced into thin sections. Embedding media include paraffin, celloidin, OCT™ compound, agar, plastics, or acrylics. In some embodiments, the sample is fixed in a formalin-based fixative and embedded in paraffin to form a formalin-fixed, paraffin-embedded (FFPE) block.

In some embodiments, if the sample is embedded in paraffin, the sample can be deparaffinized using appropriate deparaffinizing process.

In some embodiments, the biological samples are pre-treated with an enzyme inactivation composition to substantially or completely inactivate endogenous peroxidase activity. For example, some cells or tissues contain endogenous peroxidase. Using an HRP conjugated antibody may result in high, non-specific background staining. This non-specific background can be reduced by pre-treatment of the sample with an enzyme inactivation composition as disclosed herein. In some embodiments, the samples are pre-treated with hydrogen peroxide only (about 1% to about 3% by weight of an appropriate pre-treatment solution) to reduce endogenous peroxidase activity. Once the endogenous peroxidase activity has been reduced or inactivated, detection kits may be added, followed by inactivation of the enzymes present in the detection kits, as provided above. The disclosed enzyme inactivation composition and methods can also be used as a method to inactivate endogenous enzyme peroxidase activity. Additional inactivation compositions are described in U.S. Publication No. 2018/0120202, the disclosure of which is hereby incorporated by reference herein in its entirety.

### Automated Staining Systems

The RIC assays described herein may be performed on an automated staining apparatus, manually, or feature a combination of automated steps and manual steps. In some embodiments, an automated staining apparatus includes one or more reservoirs (such as for storage of the various reagents used in the labeling protocols), one or more reagent dispense units in fluid communication with the one or more reservoirs for dispensing reagent to onto a sample, a waste removal system for removing used reagents and other waste from the sample, and a control system that coordinates the actions of the one or more reagent dispense units and a waste removal system. In addition to performing labeling steps, an automated staining apparatus may be configured to perform steps ancillary to labeling (or are compatible with separate systems that perform such ancillary steps), including, but not limited to, slide baking (for adhering the sample to a slide), dewaxing (also referred to as deparaffinization), antigen retrieval, counterstaining, dehydration and clearing, and coverslipping.

Prichard, Overview of Automated Immunohistochemistry, Arch Pathol Lab Med., Vol. 138, pp. 1578-1582 (2014), the disclosure of which is hereby incorporated herein by reference in its entirety, describes several specific examples of automated staining apparatus and their various features, including the intelliPATH (Biocare Medical), WAVE (Celerus Diagnostics), DAKO OMNIS and DAKO AUTOSTAINER LINK 48 (Agilent Technologies), BENCHMARK (Ventana Medical Systems, Inc.), Leica BOND, and LAB VISION AUTOSTAINER (Thermo Scientific) automated AHC labeling systems. Additionally, Ventana Medical Systems, Inc. is the assignee of a number of United States patents disclosing systems and methods for performing automated analyses, including U.S. Pat. Nos. 5,650,327, 5,654,200, 6,296,809, 6,352,861, 6,827,901 and 6,943,029, and U.S. Published Patent Application Nos. 20030211630 and 20040052685, each of which is incorporated herein by reference in its entirety.

An automated staining apparatus typically operates on one of the following principles: (1) open individual slide labeling, in which slides are positioned horizontally and reagents are dispensed as a puddle on the surface of the slide containing a tissue sample (such as implemented on the DAKO AUTOSTAINER Link 48 (Agilent Technologies) and INTELLIPATH (Biocare Medical) labelers); (2) liquid overlay technology, in which reagents are either covered with or dispensed through an inert fluid layer deposited over the sample (such as implemented on BENCHMARK and DISCOVERY labelers); (3) capillary gap labeling, in which the slide surface is placed in proximity to another surface (which may be another slide or a coverplate) to create a narrow gap, through which capillary forces draw up and keep liquid reagents in contact with the samples (such as the labeling principles used by DAKO TECHMATE, Leica BOND, and DAKO OMNIS labelers).

Some iterations of capillary gap labeling do not mix the fluids in the gap (such as on the DAKO TECHMATE and the Leica BOND). In variations of capillary gap labeling termed dynamic gap labeling, capillary forces are used to apply sample to the slide, and then the parallel surfaces are translated relative to one another to agitate the reagents during incubation to effect reagent mixing (such as the labeling principles implemented on DAKO OMNIS slide labelers (Agilent)). In translating gap labeling, a translatable head is positioned over the slide. A lower surface of the head is spaced apart from the slide by a first gap sufficiently small to allow a meniscus of liquid to form from liquid on the slide during translation of the slide. A mixing extension having a lateral dimension less than the width of a slide extends from the lower surface of the translatable head to define a second gap smaller than the first gap between the mixing extension and the slide. During translation of the head, the lateral dimension of the mixing extension is sufficient to generate lateral movement in the liquid on the slide in a direction generally extending from the second gap to the first gap (see WO 2011/139978A1, the disclosure of which is hereby incorporated by reference herein in its entirety). It has also been proposed to use inkjet technology to deposit reagents on slides (see WO 2016/170008A1, the disclosure of which is hereby incorporated by reference herein in its entirety). This list of labeling technologies is not intended to be comprehensive, and any fully or semi-automated system or manual method for performing biomarker labeling may be incorporated into the present methods.

### Specific Binding Entities

The present disclosure employs at least three different specific binding entities, namely binding entities specific for the ER, Ki-67, and PR biomarkers.

As used herein, the phrases “specific binding agent,” “specific binding entity,” “biomarker-specific reagent,” or the like refer to any composition of matter that is capable of specifically binding to a target chemical structure associated with a cellular sample (such as a biomarker expressed by the sample, or a biomarker-specific reagent bound to the sample). Examples include antibodies and antigen binding fragments thereof, and engineered specific binding structures, including ADNECTINs (scaffold based on 10th FN3 fibronectin; Bristol-Myers-Squibb Co.), AFFIBODYs (scaffold based on Z domain of protein A from S. aureus; Affibody AB, Solna, Sweden), AVIMERs (scaffold based on domain A/LDL receptor; Amgen, Thousand Oaks, CA), dAbs (scaffold based on VH or VL antibody domain; GlaxoSmithKline PLC, Cambridge, UK), DARPins (scaffold based on Ankyrin repeat proteins; Molecular Partners AG, Zurich, CH), ANTICALINs (scaffold based on lipocalins; Pieris AG, Freising, DE), NANOBODYs (scaffold based on VHH (camelid Ig); Ablynx N/V, Ghent, BE), TRANS-BODYs (scaffold based on Transferrin; Pfizer Inc., New York, NY), SMIPs (Emergent Biosolutions, Inc., Rockville, MD), and TETRANECTINs (scaffold based on C-type lectin domain (CTLD), tetranectin; Borean Pharma A/S, Aarhus, DK). Descriptions of such engineered specific binding structures are reviewed by Wurch et al., Development of Novel Protein Scaffolds as Alternatives to Whole Antibodies for Imaging and Therapy: Status on DISCOVERY Research and Clinical Validation, Current Pharmaceutical Biotechnology, Vol. 9, pp. 502-509 (2008), the content of which is incorporated by reference.

In some embodiments, the biomarker-specific reagents specific for the ER, Ki-67, and PR biomarkers are antibodies, such as monoclonal antibodies (such as mouse monoclonal or rabbit monoclonal antibodies).

Non-limiting examples of human Ki-67 biomarker-specific antibodies include those belonging the MIB@-family such as MIB-1, MIB-2, MIB-5, MIB-7, MIB-21, and MIB-24. Examples of human Ki-67 biomarker-specific antibodies include an anti-Ki-67 (30-9) monoclonal antibody available from Ventana Medical Systems, Inc. (Tucson, AZ). Other examples of human Ki-67 biomarker-specific antibodies include clone 30-9 (Roche) and clone Ki-67 (BioLegend).

Non-limiting examples of human ER biomarker-specific antibodies include an anti-ER (SP1) monoclonal antibody available from Ventana Medical Systems, Inc. (Tucson, AZ). Another example of a human ER biomarker-specific antibodies includes the anti-ER monoclonal antibody 1D5 available from Invitrogen. A high-affinity monoclonal antibody for recognizing the ER receptor is described in U.S. Pat. No. 7,569,675, the disclosure of which is hereby incorporated by reference herein in its entirety.

Non-limiting examples of human ER biomarker-specific antibodies include an anti-PR (1E2) monoclonal antibody available from Ventana Medical Systems, Inc. (Tucson, AZ). A high-affinity monoclonal antibody for recognizing the PR receptor is described in U.S. Pat. No. 7,569,675, the disclosure of which is hereby incorporated by reference herein in its entirety.

### Detection Reagents and Detectable Moieties

Detection of the biomarker in the sample is achieved by depositing a detectable moiety in close proximity to the biomarker-specific reagent bound to the sample. In some embodiments, the detectable moiety is directly or indirectly conjugated to the biomarker-specific reagent (e.g., a monoclonal antibody, including any of those described herein), and thus is deposited on the sample upon binding of the biomarker-specific reagent to its target (generally referred to as a direct labeling method). In other embodiments, deposition of the detectable moiety is effected by the applying a set of detection reagents to the sample after the application of the biomarker-specific reagent, wherein the detection reagents bind to or otherwise react with the biomarker-specific reagent in a manner the effects deposition of the detectable moiety (generally referred to as an indirect labeling method).

By way of example, and in other embodiments, the biomarker-specific reagent (e.g., the human ER biomarker-specific antibody, the human Ki-67 biomarker-specific antibody, and the human PR biomarker-specific antibody) does not include a detectable moiety. In these embodiments, the sample is then contacted with a set of detection reagents that interact with the specific binding agent to facilitate deposition of a detectable moiety (e.g., a chromogen) onto or in close proximity the biomarker, thereby generating a detectable signal localized to the biomarker.

In some embodiments in which an indirect method is used, the detectable moiety is deposited via an enzymatic reaction localized to the biomarker-specific reagent (e.g., monoclonal antibody). Suitable enzymes for such reactions are well-known and include, but are not limited to, oxidoreductases, hydrolases, and peroxidases. Specific enzymes explicitly included are horseradish peroxidase (HRP), alkaline phosphatase (AP), acid phosphatase, glucose oxidase, p-galactosidase, P-glucuronidase, and P-lactamase. The enzyme may be directly conjugated to the biomarker-specific reagent (e.g., monoclonal antibody), or may be indirectly associated with the biomarker-specific reagent (e.g., monoclonal antibody) via a labeling conjugate. As used herein, a “labeling conjugate” comprises:
(a) a specific detection reagent; and
(b) an enzyme conjugated to the specific detection reagent, wherein the enzyme is reactive with a chromogenic substrate, a signaling conjugate, and/or an enzyme-reactive dye under appropriate reaction conditions to effect in situ generation of the dye and/or deposition of the dye on the tissue sample.

In non-limiting examples, the specific detection reagent of the labeling conjugate may be a secondary detection reagent (such as a species-specific secondary antibody bound to a primary antibody, an anti-hapten antibody bound to a hapten-conjugated primary antibody, or a biotin-binding protein bound to a biotinylated primary antibody), a tertiary detection reagent (such as a species-specific tertiary antibody bound to a secondary antibody, an anti-hapten antibody bound to a hapten-conjugated secondary antibody, or a biotin-binding protein bound to a biotinylated secondary antibody),or other such arrangements. An enzyme thus localized to the sample-bound biomarker-specific reagent (e.g., monoclonal antibody) can then be used in a number of schemes to deposit a detectable moiety.

In non-limiting examples, the specific detection reagent of the labeling conjugate may be a secondary detection reagent (such as a species-specific secondary antibody bound to a primary antibody, an anti-hapten antibody bound to a hapten-conjugated primary antibody, or a biotin-binding protein bound to a biotinylated primary antibody), a tertiary detection reagent (such as a species-specific tertiary antibody bound to a secondary antibody, an anti-hapten antibody bound to a hapten-conjugated secondary antibody, or a biotin-binding protein bound to a biotinylated secondary antibody),or other such arrangements. An enzyme thus localized to the sample-bound biomarker-specific reagent can then be used in a number of schemes to deposit a detectable moiety. In some cases, the enzyme reacts with a chromogenic compound/substrate.

In yet other embodiments, the detectable moiety is deposited via a signaling conjugate comprising a latent reactive moiety configured to react with the enzyme to form a reactive species that can bind to the sample or to other detection components. These reactive species are capable of reacting with the sample proximal to their generation, i.e. near the enzyme, but rapidly convert to a non-reactive species so that the signaling conjugate is not deposited at sites distal from the site at which the enzyme is deposited. Examples of latent reactive moieties include: quinone methide (QM) analogs, such as those described at WO2015124703A1, and tyramide conjugates, such as those described at, WO2012003476A2, each of which is hereby incorporated by reference herein in its entirety. In some examples, the latent reactive moiety is directly conjugated to a dye, such as N,N′-biscarboxypentyl-5,5′-disulfonato-indo-dicarbocyanine (Cy5), 4-(dimethylamino) azobenzene-4′-sulfonamide (DABSYL), tetramethylrhodamine (DISCO Purple), and Rhodamine 110 (Rhodamine). In other examples, the latent reactive moiety is conjugated to one member of a specific binding pair, and the dye is linked to the other member of the specific binding pair. In other examples, the latent reactive moiety is linked to one member of a specific binding pair, and an enzyme is linked to the other member of the specific binding pair, wherein the enzyme is (a) reactive with a chromogenic substrate to effect generation of the dye, or (b) reactive with a dye to effect deposition of the dye (such as DAB). Examples of specific binding pairs include: (1) a biotin or a biotin derivative (such as desthiobiotin) linked to the latent reactive moiety, and a biotin-binding entity (such as avidin, streptavidin, deglycosylated avidin (such as NEUTRAVIDIN), or a biotin binding protein having a nitrated tyrosine at its biotin binding site (such as CAPTAVIDIN)) linked to a dye or to an enzyme reactive with a chromogenic substrate or reactive with a dye (for example, a peroxidase linked to the biotin-binding protein when the dye is DAB); and (2) a hapten linked to the latent reactive moiety, and an anti-hapten antibody linked to a dye or to an enzyme reactive with a chromogenic substrate or reactive with a dye (for example, a peroxidase linked to the biotin-binding protein when the dye is DAB).

Any detection reagents or detectable moieties compatible with multiplex affinity histochemistry or affinity cytochemistry, e.g., immunohistochemistry, may be utilized in the methods of the present disclosure. In some embodiments, the detectable moiety is a molecule detectable via brightfield microscopy. Non-limiting examples of brightfield detectable moieties compatible with IHC, including multiplex IHC, and methodologies of using the brightfield detectable moieties are disclosed in U.S. Pat. No. 10,041,950, the disclosure of which is hereby incorporated by reference herein in its entirety. Specific examples of brightfield detectable moieties (also referred to as chromogens) include, but are not limited to, diaminobenzidine (DAB), 4-(dimethylamino) azobenzene-4′-sulfonamide (DABSYL), tetramethylrhodamine, N,N′-biscarboxypentyl-5,5′-disulfonato-indo-dicarbocyanine (Cy5), and Rhodamine 110 (Rhodamine), 4-nitrophenylphospate (pNPP), fast red, bromochloroindolyl phosphate (BCIP), nitro blue tetrazolium (NBT), BCIP/NBT, fast red, AP Orange, AP blue, tetramethylbenzidine (TMB), 2,2′-azino-di-[3-ethylbenzothiazoline sulphonate](ABTS), 4-chloronaphthol (4-CN), nitrophenyl-β-D-galactopyranoside (ONPG), o-phenylenediamine (OPD), 5-bromo-4-chloro-3-indolyl-β-galactopyranoside (X-Gal), methylumbelliferyl-β-D-galactopyranoside (MU-Gal), p-nitrophenyl-α-D-galactopyranoside (PNP), 5-bromo-4-chloro-3-indolyl-β-D-glucuronide (X-Gluc), 3-amino-9-ethyl carbazol (AEC), fuchsin, iodonitrotetrazolium (INT), tetrazolium blue, or tetrazolium violet. In some embodiments, the brightfield detectable moieties are translucent. Given the translucency of the brightfield detectable moieties, different color combinations may result if two or more brightfield detectable moieties are deposited in or about the same location. In the context of staining particular biomarkers within cells or cell nuclei, the translucency of brightfield detectable moieties facilitates the detection of the different expression patterns of two or more biomarkers due to the appearance of particular colors that result from the different brightfield detectable moieties combinations.

In some embodiments, the brightfield detectable moieties is selected from TAMRA, Dabsyl, Dabcyl, Cy3, CyB, Cy3.5, Cy5, Cy5.5, Cy7, rhodamine 800 and fluorescein. In some embodiments, the brightfield detectable moieties is a conjugate comprising at least two chromogens, such as at least two chromogens selected from TAMRA, Dabsyl, Dabcyl, Cy3, CyB, Cy3.5, Cy5, Cy5.5, Cy7, rhodamine 800 and fluorescein. In some embodiments, the brightfield detectable moieties is selected from those disclosed in United States Patent Publication No. 2021/0055285, the disclosure of which is hereby incorporated by reference herein in its entirety.

Non-limiting examples of commercially available detection reagents or kits comprising detection reagents suitable for use with present methods include: VENTANA ULTRAVIEW detection systems (secondary antibodies conjugated to enzymes, including HRP and AP); VENTANA IVIEW detection systems (biotinylated anti-species secondary antibodies and streptavidin-conjugated enzymes); VENTANA OPTIVIEW detection systems (OptiView) (anti-species secondary antibody conjugated to a hapten and an anti-hapten tertiary antibody conjugated to an enzyme multimer); VENTANA Amplification kit (unconjugated secondary antibodies, which can be used with any of the foregoing VENTANA detection systems to amplify the number of enzymes deposited at the site of primary antibody binding); VENTANA OPTIVIEW Amplification system (Anti-species secondary antibody conjugated to a hapten, an anti-hapten tertiary antibody conjugated to an enzyme multimer, and a tyramide conjugated to the same hapten. In use, the secondary antibody is contacted with the sample to effect binding to the primary antibody. Then the sample is incubated with the anti-hapten antibody to effect association of the enzyme to the secondary antibody. The sample is then incubated with the tyramide to effect deposition of additional hapten molecules. The sample is then incubated again with the anti-hapten antibody to effect deposition of additional enzyme molecules. The sample is then incubated with the detectable moiety to effect dye deposition); VENTANA DISCOVERY, DISCOVERY OMNIMAP, DISCOVERY ULTRAMAP anti-hapten antibody, secondary antibody, chromogen, fluorophore, and dye kits, each of which are available from Ventana Medical Systems, Inc. (Tucson, Arizona); POWERVISION and POWERVISION+ IHC Detection Systems (secondary antibodies directly polymerized with HRP or AP into compact polymers bearing a high ratio of enzymes to antibodies); DAKO ENVISION™+ System (enzyme labeled polymer that is conjugated to secondary antibodies); ULTRAPLEX Multiplex Chromogenic IHC Technology from CELL IDx (hapten-labeled primary antibodies combined with enzyme-labeled or fluor-labeled anti-hapten secondary antibodies).

Commercial brightfield detectable moieties include DISCOVERY Red, DISCOVERY Yellow, DISCOVERY Blue, DISCOVERY Purple, DISCOVERY Silver, DISCOVERY Teal, and DISCOVERY Green, each of which are available from Roche Diagnostics.

Yet other suitable detectable conjugates including different detectable moieties are disclosed in PCT Publication No. WO/2022/043491, the disclosure of which is hereby incorporated by reference in its entirety. For instance, PCT Publication No. WO/2022/043491 discloses detectable moieties having different “core” structures, e.g., a coumarin core, a phenoxazinone core, a 4-Hydroxy-3-phenoxazinone core, a 7-amino-4-Hydroxy-3-phenoxazinone core, a thioninium core, a phenoxazine core, a phenoxathiin-3-one core, or a xanthene core. Any of these detectable moieties may be suitable for labeling ER, Ki-67, and/or PR biomarkers.

Other detection reagents, detectable moieties, and detection strategies are described in U.S. Pat. Nos. 11,249,085, 11,249,085, and 10,168,336; and in United States Patent Application Publication No. 2012/0171668, the disclosures of which are hereby incorporated by reference herein in their entireties.

Typically, wash steps are performed between application of different reagents to prevent unwanted non-specific labeling of tissues. For instance, washing steps may be performed after each of these pre-processing steps by applying one or more passes of a wash buffer. By way of example, wash buffers typically are neutrally buffered saline solutions, which may also contain small amounts of detergent. Non-limiting examples of wash buffers include, for example, Phosphate Buffered Saline (PBS), PBS-Tween20, Tris Buffered Saline (TBS), TBS-Tween20 (polysorbate 20), Tris-HCl, Tris-HC-Tween20, Phosphate Buffer (PB), AP Buffer, and the like.

### Counterstaining and Morphological Staining

In some embodiments, the samples may be counterstained to assist in identifying morphologically relevant areas, either manually or automatically. Examples of counterstains include chromogenic nuclear counterstains, such as hematoxylin (stains from blue to violet), Methylene blue (stains blue), toluidine blue (stains nuclei deep blue and polysaccharides pink to red), nuclear fast red (also called Kernechtrot dye, stains red), and methyl green (stains green); and non-nuclear chromogenic stains, such as eosin (stains pink). In some embodiments, the counterstain is a non-nuclear chromogenic stain, such as eosin (stains pink); fluorescent nuclear stains, including 4′, 6-diamino-2-pheylindole (DAPI, stains blue), propidium iodide (stains red), Hoechst stain (stains blue), nuclear green DCS1 (stains green), nuclear yellow (Hoechst S769121, stains yellow under neutral pH and stains blue under acidic pH), DRAQ5 (stains red), DRAQ7 (stains red); fluorescent non-nuclear stains, such as fluorophore-labelled phalloidin, (stains filamentous actin, color depends on conjugated fluorophore).

In some embodiments, a serial section of the biomarker-labeled section may be morphologically stained, which can be used to identify particular regions of interest in which to evaluate the biomarker-stained sample. Many morphological stains are known, including but not limited to, hematoxylin and eosin (H&E) stain and Lee's Stain (Methylene Blue and Basic Fuchsin). In a specific embodiment, at least one serial section of each biomarker-labeled slide is H&E stained. Any method of applying H&E stain may be used, including manual and automated methods. In an embodiment, at least one section of the sample is an H&E stained sample stained on an automated staining system. Automated systems for performing H&E staining typically operate on one of two staining principles: batch staining (also referred to as “dip 'n dunk”) or individual slide staining. Batch stainers generally use vats or baths of reagents in which many slides are immersed at the same time. Individual slide stainers, on the other hand, apply reagent directly to each slide, and no two slides share the same aliquot of reagent. Examples of commercially available H&E stainers include the VENTANA HE 600 series H&E stainers (individual slide stainer) from Roche; the DAKO COVERSTAINER from Agilent Technologies (batch stainer); the LEICA ST4020 Small Linear Stainer, LEICA ST5020 MULTISTAINER, and the LEICA ST5010 AUTOSTAINER XL series H&E stainers from Leica Biosystems Nussloch GmbH (batch stainers).

### Multiplex Affinity Histochemical or Affinity Cytochemical Methods

In some embodiments, the affinity histochemical or affinity cytochemical assay, e.g., IHC assay, of the present disclosure is provided in a multiplex format.

A multiplex IHC format involves affinity staining of multiple biomarkers in a single sample where at least some of the biomarkers are differentially labeled. For instance, a duplex IHC assay for two distinct biomarkers in the same sample (e.g., ER and PR; ER and Ki-67; Ki-67 and PR) and where each of the two biomarkers are stained a different brightfield detectable moieties would be considered a “multiplex IHC assay.” Likewise, a triplex IHC assay for three biomarkers where each of the three biomarkers are stained with a different brightfield detectable moieties would also be considered a “multiplex IHC assay.” The detection reagents including any detectable moieties used in such a method should be compatible multiplex immunohistochemistry.

A multiplex IHC assay may be performed to stain a sample for the expression of the ER, Ki-67, and PR biomarkers (in any order or in a pre-determined order). In some embodiments, a triplex method is provided in which human ER, Ki-67, and PR markers are differentially stained in the same sample, such as differentially stained with three different brightfield detectable moieties, e.g., three different brightfield detectable moieties having different detectable and/or different distinguishable signals.

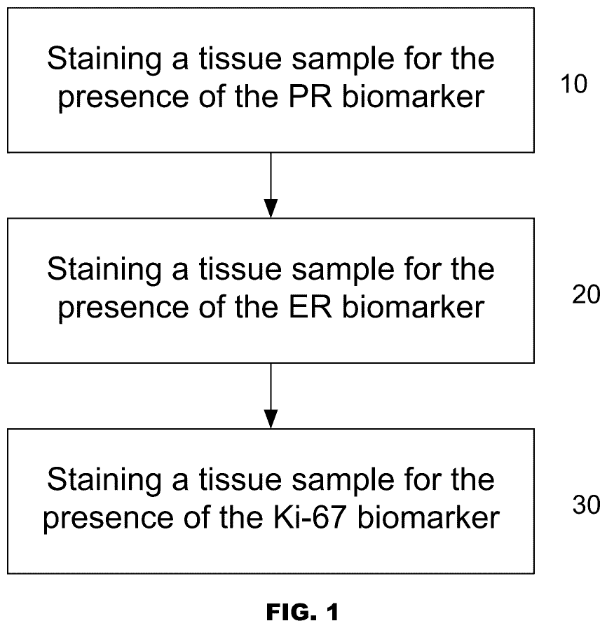

With reference to FIG. 1, in some embodiments, the triplex IHC assay of the present disclosure includes staining a tissue sample for the presence the PR biomarker (step 10), for the presence of the ER biomarker (step 20), and for the presence of the Ki-67 biomarker (step 30). While FIG. 1 depicts that the sequential staining of the PR, ER, and Ki-67 biomarkers, the biomarkers may be stained in any order. For instance, the ER marker may be stained first, the PR marker stained second and the Ki-67 biomarker stained third. Likewise, the ER marker may be stained first, the Ki-67 marker stained second and the ER biomarker stained third. Similarly, the PR marker may be stained first, the Ki-67 marker stained second and the ER biomarker stained third. Yet alternatively, the Ki-67 marker may be stained first, the ER marker stained second and the PR biomarker stained third. Yet even more alternatively, the Ki-67 marker may be stained first, the PR marker stained second and the ER biomarker stained third.

In some embodiments, the method includes the steps of (i) contacting a biological sample with a first human biomarker specific agent to one of ER, Ki-67, or PR; and (ii) contacting the biological sample with first detection reagents for labeling the first human biomarker specific agent with a first brightfield detectable moiety. In some embodiments, the first human biomarker specific agent is a first primary monoclonal antibody specific to one of ER, Ki-67, or PR. In some embodiments, the first detection reagents include a secondary antibody specific to the first primary monoclonal antibody and wherein the secondary antibody is conjugated to first enzyme, e.g., horseradish peroxidase or alkaline phosphatase. In some embodiments, the first detection reagents include a conjugate comprising a first brightfield detectable moiety such that when the first enzyme acts on the conjugate comprising the first brightfield detectable moiety, the first brightfield detectable moiety is deposited onto or in close proximity to the one of the ER, Ki-67, or PR biomarker. In some embodiments, the first enzyme is inactivated prior to introducing further detection reagents.

In some embodiments, the method further includes the steps of (iii) contacting a biological sample with a second human biomarker specific agent to another one of ER, Ki-67, or PR; and (iv) contacting the biological sample with second detection reagents for labeling the second human biomarker specific agent with a second brightfield detectable moiety. In some embodiments, the second human biomarker specific agent is a second primary monoclonal antibody specific to another one of ER, Ki-67, or PR. In some embodiments, the second detection reagents include a secondary antibody specific to the second primary monoclonal antibody and wherein the secondary antibody is conjugated to a second enzyme, e.g., horseradish peroxidase or alkaline phosphatase. In some embodiments, the second detection reagents include a conjugate comprising a second brightfield detectable moiety such that when the second enzyme acts on the conjugate comprising the second brightfield detectable moiety, the second brightfield detectable moiety is deposited onto or in close proximity to another one of the ER, Ki-67, or PR biomarker. In some embodiments, the second brightfield detectable moiety is different than the first brightfield detectable moiety. In some embodiments, the second enzyme is inactivated prior to introducing further detection reagents.

In some embodiments, the method includes the steps of (v) contacting a biological sample with a third human biomarker specific agent to a third one of ER, Ki-67, or PR; and (vi) contacting the biological sample with third detection reagents for labeling the third human biomarker specific agent with a third brightfield detectable moiety. In some embodiments, the third human biomarker specific agent is a third primary monoclonal antibody specific to the third one of ER, Ki-67, or PR. In some embodiments, the third detection reagents include a secondary antibody specific to the third primary monoclonal antibody and wherein the secondary antibody is conjugated to a third enzyme, e.g., horseradish peroxidase or alkaline phosphatase. In some embodiments, the third detection reagents include a conjugate comprising a third brightfield detectable moiety such that when the third enzyme acts on the conjugate comprising the third brightfield detectable moiety, the third brightfield detectable moiety is deposited onto or in close proximity to the third one of the ER, Ki-67, or PR biomarker. In some embodiments, the third brightfield detectable moiety is different than the first and second brightfield detectable moiety.

In the context of a multiplex assay where multiple brightfield detectable moieties are detected sequentially; it is desirable to inactivate any reagent or endogenous enzymes between successive brightfield detectable moiety detection steps. As a result, it is believed that enzymes present in any one brightfield detectable moiety detection step will not interfere with those in a later brightfield detectable moiety detection step. This in turn is believed to improve upon the visualization and detection of the different brightfield detectable moieties used in the multiplex assay. Any enzyme inactivation composition known in the art may be used for this purpose. In some embodiments, an enzyme inactivation composition is applied to inactivate the reagent or endogenous enzymes after each detection step. Exemplary enzyme inactivation compositions are disclosed in U.S. Pat. No. 11,162,877, the disclosure of which is hereby incorporated by reference herein in its entirety.

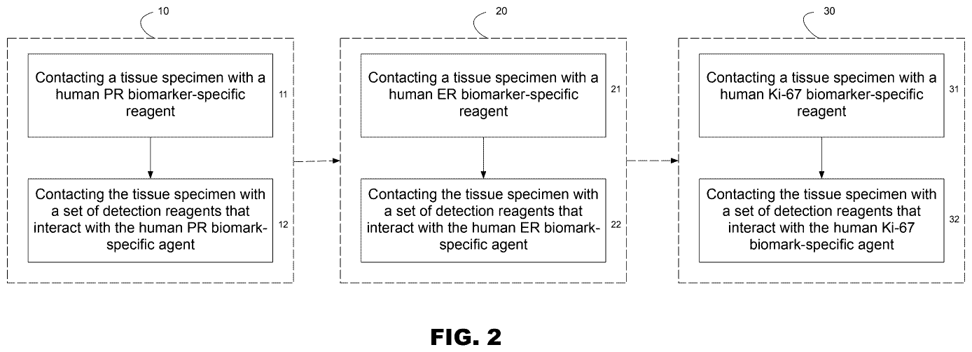

With reference to FIG. 2, in some embodiments, the method includes the steps of (i) contacting a biological sample with a human PR biomarker specific agent (step 11); and (ii) contacting the biological sample with first detection reagents for labeling the human PR biomarker specific agent with a first brightfield detectable moiety (step 12) so as to stain the biological sample for the presence of the PR biomarker (step 10). In some embodiments, the first detection reagents include a secondary antibody specific to the human PR biomarker specific agent and wherein the secondary antibody is conjugated to first enzyme, e.g., horseradish peroxidase or alkaline phosphatase. In some embodiments, the first detection reagents include a conjugate comprising a first brightfield detectable moiety such that when the first enzyme acts on the conjugate comprising the first brightfield detectable moiety, the first brightfield detectable moiety is deposited onto or in close proximity to the PR biomarker. In some embodiments, the first brightfield detectable moiety is selected from TAMRA, Dabsyl, Dabcyl, Cy3, CyB, Cy3.5, Cy5, Cy5.5, Cy7, rhodamine 800 and fluorescein. In some embodiments, the first brightfield detectable moiety is a conjugate comprising at least two chromogens, such as at least two chromogens selected from TAMRA, Dabsyl, Dabcyl, Cy3, CyB, Cy3.5, Cy5, Cy5.5, Cy7, rhodamine 800 and fluorescein. In other embodiments, the first brightfield detectable moiety is selected from one of Discovery Purple, Discovery Teal, or Discovery Yellow. In some embodiments, an enzyme inactivation composition is introduced to the biological sample prior to staining for the presence of a second biomarker.

In some embodiments, the method includes the steps of (iii) contacting a biological sample with a human ER biomarker specific agent (step 21); and (iv) contacting the biological sample with second detection reagents for labeling the human ER biomarker specific agent with a second brightfield detectable moiety (step 22) so as to stain the biological sample for the presence of the ER biomarker (step 20). In some embodiments, the second detection reagents include a secondary antibody specific to the human ER biomarker specific agent and wherein the secondary antibody is conjugated to second enzyme, e.g., horseradish peroxidase or alkaline phosphatase. In some embodiments, the second detection reagents include a conjugate comprising a second brightfield detectable moiety such that when the second enzyme acts on the conjugate comprising the second brightfield detectable moiety, the second brightfield detectable moiety is deposited onto or in close proximity to the ER biomarker. In some embodiments, the second brightfield detectable moiety is selected from TAMIRA, Dabsyl, Dabcyl, Cy3, CyB, Cy3.5, Cy5, Cy5.5, Cy7, rhodamine 800 and fluorescein. In some embodiments, the second brightfield detectable moiety is a conjugate comprising at least two chromogens, such as at least two chromogens selected from TAMRA, Dabsyl, Dabcyl, Cy3, CyB, Cy3.5, Cy5, Cy5.5, Cy7, rhodamine 800 and fluorescein. In other embodiments, the second brightfield detectable moiety is selected from one of Discovery Purple, Discovery Teal, or Discovery Yellow. In some embodiments, an enzyme inactivation composition is introduced to the biological sample prior to staining for the presence of a third biomarker.

In some embodiments, the method includes the steps of (v) contacting a biological sample with a human Ki-67 biomarker specific agent (step 31); and (vi) contacting the biological sample with third detection reagents for labeling the human Ki-67 biomarker specific agent with a third brightfield detectable moiety (step 32) so as to stain the biological sample for the presence of the Ki-67 biomarker (step 30). In some embodiments, the third detection reagents include a secondary antibody specific to the human Ki-67 biomarker specific agent and wherein the secondary antibody is conjugated to third enzyme, e.g., horseradish peroxidase or alkaline phosphatase. In some embodiments, the third detection reagents include a conjugate comprising a third brightfield detectable moiety such that when the third enzyme acts on the conjugate comprising the third brightfield detectable moiety, the third brightfield detectable moiety is deposited onto or in close proximity to the Ki-67 biomarker. In some embodiments, the third brightfield detectable moiety is selected from TAMRA, Dabsyl, Dabcyl, Cy3, CyB, Cy3.5, Cy5, Cy5.5, Cy7, rhodamine 800 and fluorescein. In some embodiments, the third brightfield detectable moiety is a conjugate comprising at least two chromogens, such as at least two chromogens selected from TAMRA, Dabsyl, Dabcyl, Cy3, CyB, Cy3.5, Cy5, Cy5.5, Cy7, rhodamine 800 and fluorescein. In other embodiments, the third brightfield detectable moiety is selected from one of Discovery Purple, Discovery Teal, or Discovery Yellow. As noted above, steps 10, 20, and 30 may be performed in any order.

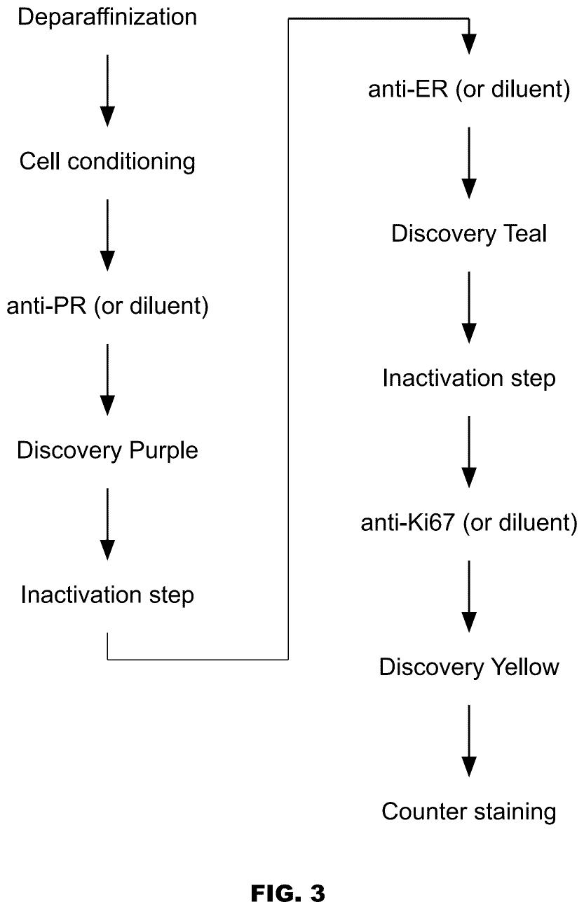

An exemplary method of staining a tissue sample for the presence of PR, ER, and Ki-67 is depicted in FIG. 3.

### Staining Assessment/Scoring

Subsequent to staining, the stained sample may be evaluated for the expression of the ER, Ki-67, and PR biomarkers. In some embodiments, the stained sample is evaluated for the colocalization of expression of the ER, Ki-67, and PR biomarker in cells or cell nuclei. In some embodiments, the staining assessment/scoring is conducted manually by a pathologist. In other embodiments, a digital pathology system is utilized to score the stained sample, such as automatically scoring the stained sample.

In some embodiments, the present disclosure comprises contacting the sample with first, second, and third detection reagents, the first detection reagent comprising components to label a first of ER, Ki-67, or PR with a first brightfield detectable moiety, the second detection reagent comprising components to label a second of ER, Ki-67, or PR with a second brightfield detectable moiety, and the third detection reagent comprising components to label a third of ER, Ki-67, or PR with a third brightfield detectable moiety, where each of the first, second, and third brightfield detectable moiety provide different first, second, and third detectable signals; and detecting a colocalization of the different first, second, and third detectable signals (given the translucency of the brightfield detectable moieties) thereby identifying cells or cell nuclei positive for each of ER, Ki-67, and PR.

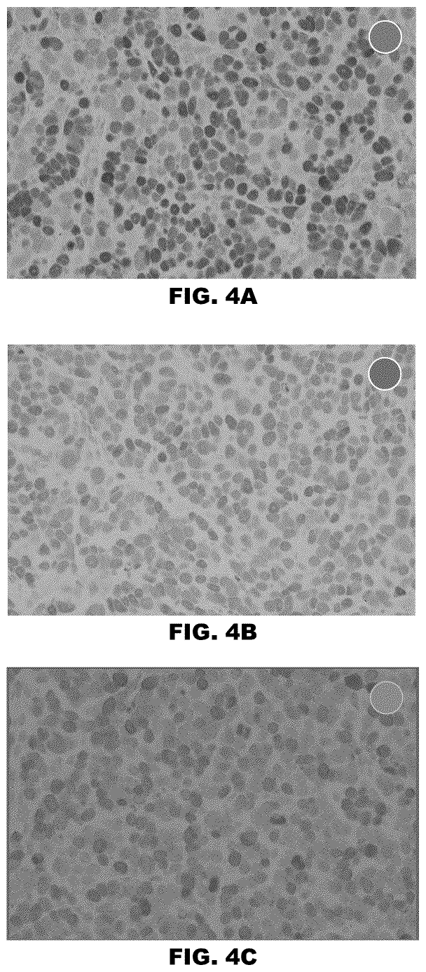

FIGS. 4A-4C illustrate individual tissue samples each stained in a simplex IHC assay with a different brightfield detectable moiety for the presence of one of the ER, PR, or Ki-67 biomarkers. FIGS. 4A-4C illustrate that different signals (e.g., different colors) may be observed for each differentially stained sample.

When a tissue sample is stained in a triplex assay for the presence of the ER, PR, and Ki-67 biomarkers as described herein, cells or cell nuclei that express each of the three biomarkers include a combination of each of the separate signals (e.g., each of the different colors) used to stain the individual biomarkers. These different signals or colors that are colocalized to a single cell or a nucleus of a single cell may result in a mixing of the separate signals or colors. These colocalized signals or colors may be interpreted during manual or automated scoring as cells that are ER+, PR+, and Ki-67 positive. For instance, a the colocalization of a yellow signal and a blue signal may result in a green signal which may be visually observed by a pathologist (given the translucency of brightfield detectable moieties utilized). Likewise, the colocalization of yellow, blue, and purple signals may result in yet a different color signal which may be turn interpreted by a pathologist.

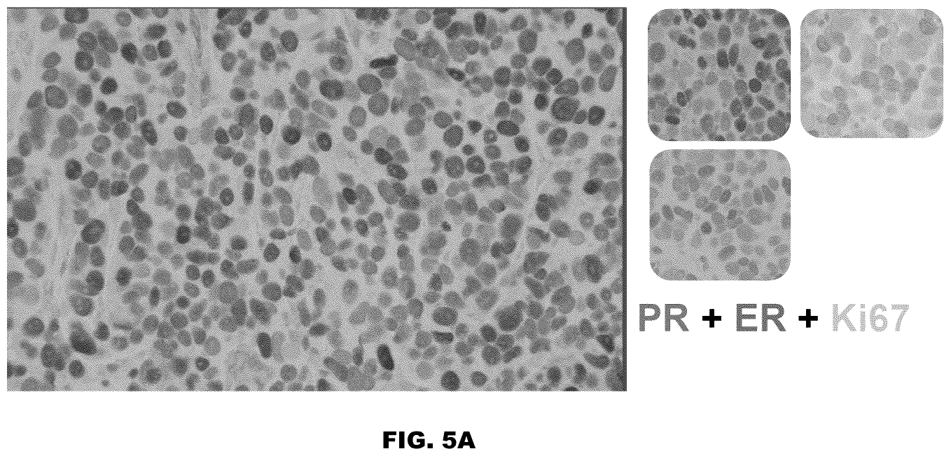

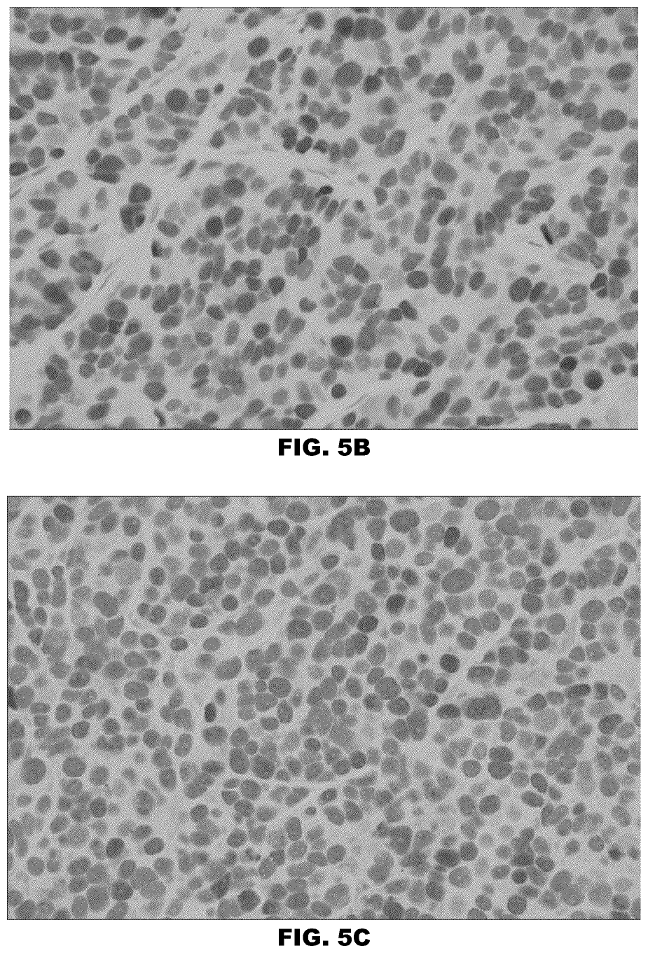

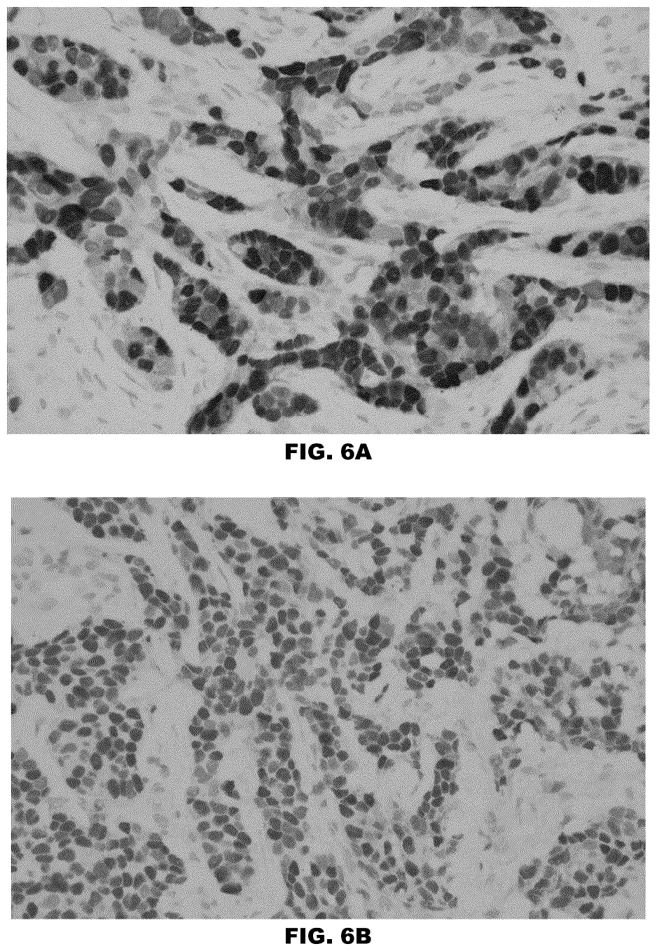

This is illustrated, for instance, in FIG. 5A, which shows cells or cell nuclei stained with a first color (purple), cells or cell nuclei stained with a second color (teal), cells or cell nuclei stained with a third color (yellow), and cells or cell nuclei stained with a combination of each of the first, second, and third colors. The cells or cell nuclei stained with the combination of each of the first, second, and third colors are indicative of cells or cell nuclei that express each of the three biomarkers. This is further illustrated in FIGS. 5B, 5C, 6A, and 6B which illustrate cells having (i) a single stain; (ii) two stains colocalized; and (iii) three stains colocalized. In some embodiments, it is the cells that contain the three colocalized stains that are scored and/or used for downstream analysis.

In some embodiments, a pathologist will identify one or more regions of interest in a tissue sample differentially stained for the presence of the ER, PR, and Ki-67 biomarkers and manually determine the number of cells or cell nuclei which include a colocalization of the three biomarkers (such as based on a manual assessment of the colocalization of the three different signals or colors).

In other embodiments, the biological sample differentially stained in the triplex assay may be evaluated using a digital pathology system. There are two basic components of digital pathology systems: (1) a scanning or image acquisition system for generating digital images of a stained sample; and (2) an image analysis system for identifying and quantifying specific features within the generated digital images.

An image acquisition system may include a scanning platform such as a slide scanner that can scan the stained slides at 20×, 40×, or other magnifications to produce high resolution whole-slide digital images, including for example slide scanners. In some embodiments, a slide scanner includes at least: (1) a microscope with lens objectives, (2) a light source (such as halogen, light emitting diode, white light, and/or multispectral light sources, depending on the dye), (3) robotics to move glass slides around (or to move the optics around the slide), (4) one or more digital cameras for image capture, (5) a computer and associated software to control the robotics and to manipulate, manage, and view digital slides. In some embodiments, digital data at a number of different X-Y locations (and in some cases, at multiple Z planes) on the slide are captured by the camera's charge-coupled device (CCD), and the images are joined together to form a composite image of the entire scanned surface. Common methods to accomplish this include: (1) Tile based scanning, in which the slide stage or the optics are moved in very small increments to capture square image frames, which overlap adjacent squares to a slight degree. In some embodiments, the captured squares are then automatically matched to one another to build the composite image; and (2) Line-based scanning, in which the slide stage moves in a single axis during acquisition to capture a number of composite image “strips.” In some embodiments, the image strips can then be matched with one another to form the larger composite image.

A detailed overview of various scanners (both fluorescent and brightfield) can be found at Farahani et al., Whole slide imaging in pathology: advantages, limitations, and emerging perspectives, Pathology and Laboratory Medicine Int'l, Vol. 7, p. 23-33 (June 2015), the disclosure of which is incorporated by reference in its entirety. Examples of commercially available slide scanners include: 3DHistech PANNORAMIC SCAN II; DigiPath PATHSCOPE; Hamamatsu NANOZOOMER RS, HT, and XR; Huron TISSUESCOPE 4000, 4000XT, and HS; Leica SCANSCOPE AT, AT2, CS, FL, and SCN400; Mikroscan D2; Olympus VS120-SL; Omnyx VL4, and VL120; PerkinElmer LAMINA; Philips ULTRA-FAST SCANNER; Sakura Finetek VISIONTEK; Unic PRECICE 500, and PRECICE 600×; and Zeiss AXIO SCAN.Z1. In some embodiments, the scanning device is a digital pathology device as disclosed any of U.S. Pat. No. 9,575,301; U.S. Patent Application Publication No. 2014/0178169; U.S. Pat. No. 9,575,301; U.S. Patent Application Publication No. 2014/0178169; United States Patent Publication Nos. 2021/0092308; and/or U.S. Patent Application Publication No. 2021/0088769, the content of each of which is incorporated by reference in its entirety.

Exemplary commercially available image analysis software packages include VENTANA VIRTUOSO software suite (Ventana Medical Systems, Inc.); TISSUE STUDIO, DEVELOPER XD, and IMAGE MINER software suites (Definiens); BIOTOPIX, ONCOTOPIX, and STEREOTOPIX software suites (Visiopharm); and the HALO platform (Indica Labs, Inc.).

A sample stained in a triplex IHC in accordance with the present disclosure may be imaged on a scanner system to generate a high-quality digital image of the stained sample. The digital image is then analyzed by the image analysis system to identify and classify one or more relevant objects in the sample.

In some embodiments, the triplex stained image is first unmixed to provide different color channel images, where each different color channel image shows the contribution of one of the three brightfield detectable moieties used in the triplex assay. Unmixing, also referred to as deconvolution, essentially separates an image having a stain mixture into the contribution of the individual single stains allowing the individual stain components to be evaluated separately. By applying deconvolution methods, individual cells and/or regions of the sample can be categorized on the basis of multiple biomarkers. Thus, for example, in an ER, Ki-67 and PR triplex assay, each tumor nuclei may be categorized on the basis of including contributions from the ER, Ki-67- and PR-associated stains. Exemplary brightfield deconvolution methods are disclosed at, for example in PCT/EP2015/061226, PCT/EP2015/067384, PCT/EP2016/081329, PCT/EP2018/070956.

In some embodiments, the image analysis may identify all cells in one or more tumor regions of interest and then classify the cells as expressing one, two, or all three of the ER, PR, and/or Ki-67 biomarkers, such as by identifying contributions of the different brightfield detectable moiety signals in each cell or cell nucleus. In some embodiments, the identified contributions of the different brightfield detectable moiety signals in cells or cell nuclei may be each compared against predetermined threshold signal amounts, e.g., intensity amounts. In some embodiments, if the contributions of any of the different brightfield detectable moiety signals exceeds the predetermined threshold signal amounts, the cell or cell nucleus may be classified as being positive for a particular marker.

After assessing the staining of the sample for the expression of the ER, Ki-67, and PR biomarkers, the sample may be classified as either (i) an ER+, Ki-67+, PR+ dominant case; or (ii) an ER+, Ki-67+ dominant case.

In some embodiments, a ratio of a number of cells or cell nuclei that stain positive for all three of ER, Ki-67, and PR to the total number of cells that are (i) ER+, Ki-67+, and PR+, and (ii) ER+, Ki-67+, and PR− is calculated, such as set forth below:

$$[ E {R}^{+} , Ki - {67}^{+} , {PR}^{+} ] / ( [ E {R}^{+} , Ki - {67}^{+} , {PR}^{+} ] + [ E {R}^{+} , Ki - {67}^{+} , {PR}^{-} ] )$$

This calculated ratio is then compared to a predetermined threshold value. If the calculated ratio is greater than or equal to the predetermined threshold value, the stained sample is assessed as an ER+, Ki-67+, PR+ dominant case. If, on the other hand, the calculated ratio is less than the predetermined threshold value, the stained sample is assessed as an ER+, Ki-67+ dominant case.

In some embodiments, the predetermined threshold value is 35%. In some embodiments, the predetermined threshold value is 40%. In some embodiments, the predetermined threshold value is 41%. In some embodiments, the predetermined threshold value is 42%. In some embodiments, the predetermined threshold value is 43%. In some embodiments, the predetermined threshold value is 44%. In some embodiments, the predetermined threshold value is 45%. In some embodiments, the predetermined threshold value is 46%. In some embodiments, the predetermined threshold value is 47%. In some embodiments, the predetermined threshold value is 48%. In some embodiments, the predetermined threshold value is 49%. In some embodiments, the predetermined threshold value is 50%. In some embodiments, the predetermined threshold value is 51%. In some embodiments, the predetermined threshold value is 52%. In some embodiments, the predetermined threshold value is 53%. In some embodiments, the predetermined threshold value is 54%. In some embodiments, the predetermined threshold value is 55%. In some embodiments, the predetermined threshold value is 55%. In some embodiments, the predetermined threshold value is 60%. In some embodiments, the predetermined threshold value is 61%. In some embodiments, the predetermined threshold value is 62%. In some embodiments, the predetermined threshold value is 63%. In some embodiments, the predetermined threshold value is 64%. In some embodiments, the predetermined threshold value is 65%.

An example of an ER+, Ki-67+, PR+ dominant case is illustrated in FIG. 6A. An example of an ER+, Ki-67+ dominant case is illustrated in FIG. 6B. Notably, FIG. 6A shows the colocalization of three different signals or colors in cells or cell nuclei. On the other hand, FIG. 6B shows the colocalization of two different signals or colors in cells (where the colocalized cells appear green due to the colocalization of a blue stain (ER+) and a yellow stain (Ki-67+).

### Methods of Treatment Based on the Assessment of ER, KI-67, and PR

The assessment of the ER, Ki-67, and PR biomarkers is useful for identifying those tumors that would benefit from administration of one or more therapeutics, such as one or more hormone therapy drugs (e.g., a selective estrogen receptor modulator (SERM), a selective estrogen receptor degrader (SERD), and an aromatase inhibitor (Al)). Indeed, Applicant has surprisingly discovered that the ER and PR status of proliferating cancer cells (i.e., those cancer cells that are Ki-67 positive) is an effective prognostic factor in ER-positive breast cancer patients (see Example herein which shows that ER+, Ki-67+, PR+ dominant patients had significantly better clinical outcomes than ER+, Ki-67+ dominant patients with hormone therapy among ER-positive and HER2-negative breast cancer patient populations).

In some embodiments, the present disclosure identifies those tumors that would benefit from administration of one or more therapeutics based on those cells or cell nuclei positively expressing each of ER, Ki-67, and PR. In some embodiments, if the sample is classified as an ER+, Ki-67+, PR+ dominant case, the sample may be further classified as likely to respond to therapy, such as hormone therapy. In other embodiments, if the sample is classified as an ER+, Ki-67+ dominant case, the sample may be further classified as not likely to respond to therapy, such as hormone therapy. Patients identified as likely to be responsive to therapy may then be administered one or more appropriate therapeutic agents. For instance, patients identified as likely to be responsive to therapy may then be administered a particular hormone therapy according to manufacturer's instructions and recommended treatment course. Patients identified as not likely to be responsive are the referred for alternative treatment courses.

In some embodiments, the assays and methods described herein may be used as a screening test to identify patients eligible for treatment with one or more of a SERM, a SERD, or an AI. In some embodiments, the assays and methods disclosed herein may be utilized to predict, or assist in predicting, a response to therapy with one or more of a SERM, a SERD, or an AI; or the results of any assay or method disclosed herein may be used to facilitate treatment with one or more of a SERM, a SERD, or an AI. Likewise, the assays and methods described herein may be used to stratify subjects into two or more classes based on their likelihood of responding to treatment with one or more of a SERM, a SERD, or an AI. For instance, based on the assessment of the expression of the components of the ER, Ki-67, and PR biomarkers, a subject in need of treatment may be stratified into a first class including those subjects likely to respond to treatment with one or more of a SERM, a SERD, or an AI; or a second class including those subjects likely not to respond to treatment with one or more of a SERM, a SERD, or an AI.

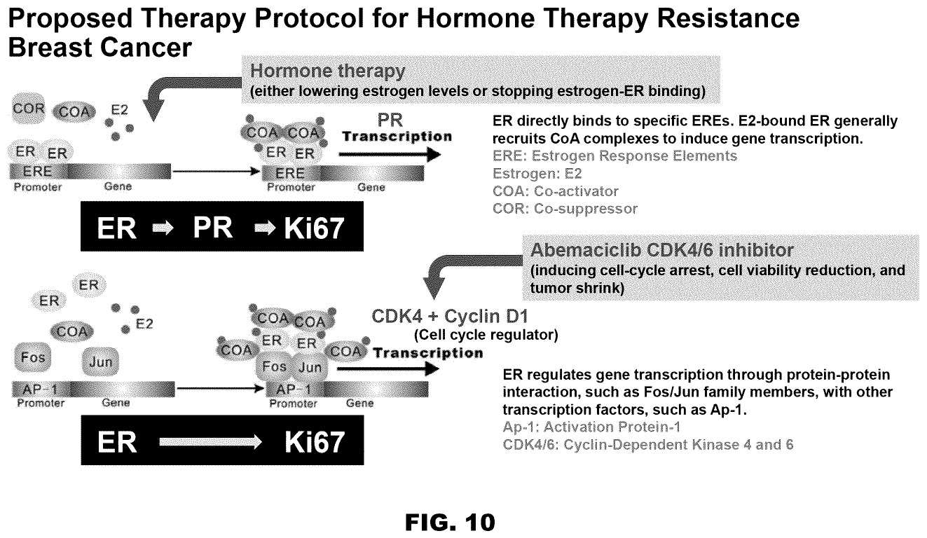

In yet other embodiments, if the sample is classified as an ER+, Ki-67+ dominant case, the sample may be further classified as likely to respond to therapy with a cyclin-dependent kinase 4 and 6 (CDK4/6) inhibitor (see, e.g., FIG. 10).

In some embodiments, the assays and methods described herein may be used as a screening test to identify patients eligible for treatment a CDK4/6 inhibitor. In some embodiments, the assays and methods disclosed herein may be utilized to predict, or assist in predicting, a response to therapy with a CDK4/6 inhibitor; or the results of any assay or method disclosed herein may be used to facilitate treatment with a CDK4/6 inhibitor. Likewise, the assays and methods described herein may be used to stratify subjects into two or more classes based on their likelihood of responding to treatment with a CDK4/6 inhibitor. For instance, based on the assessment of the expression of the components of the ER, Ki-67, and PR biomarkers, a subject in need of treatment may be stratified into a first class including those subjects likely to respond to treatment a CDK4/6 inhibitor; or a second class including those subjects likely not to respond to treatment with a CDK4/6 inhibitor.

The present disclosure is also directed to methods of selecting or identifying subjects (e.g., cancer patients) who are appropriate candidates for treatment with a therapy (e.g., with one or more hormone therapies) for treatment of cancer. Such individuals include subjects that are predicted to be responsive to the therapy (e.g., with one or more hormone therapies) and thus have an increased likelihood of benefiting from administration of the therapy relative to other patients having different characteristic(s) (e.g., non-responsiveness to the therapy). In certain embodiments an appropriate candidate is one who is reasonably likely to benefit from treatment or at least sufficiently likely to benefit as to justify administering the treatment in view of its risks and side effects. The disclosure also encompasses methods of selecting or identifying subjects (e.g., cancer patients) who are not appropriate candidates for treatment with a therapy (e.g., with one or more hormone therapies) for treatment of cancer. Such subjects include cancer patients that are predicted to be non-responsive or weakly responsive to the therapy and thus have a decreased likelihood of benefiting from administration of the therapy relative to other patients having different characteristic(s) (e.g., responsiveness to the therapy), or a low or substantially no likelihood of benefiting from such treatment, such that it may be desirable to use a different or additional treatment. In some embodiments, whether a patient is an appropriate candidate for therapy with one or more hormone therapies is determined based on an assessment of the expression of ER, Ki-67, and PR in a sample derived from the patient as described herein.

In some embodiments, the assays and methods disclosed herein may be used in the treatment of cancer (e.g., breast cancer). For instance, for those subjects whose tumor sample is assessed as an ER+, Ki-67+, PR+ dominant case, a therapeutically effective amount of one or more hormone therapy drugs may be administered. For instance, the subject in need of treatment thereof may be administered one or more of a selective estrogen receptor modulator, a selective estrogen receptor degrader, or an aromatase inhibitor.

In other embodiments, for those subjects whose tumor sample is assessed as an ER+, Ki-67+ dominant case, a therapeutically effective amount of one or more CDK4/6 inhibitors may be administered (e.g., Abemaciclib).

In yet other embodiments, the present disclosure is directed to methods of treating subjects, e.g., a human patients, having cancer comprising: (a) selecting a subject that is a suitable candidate for treatment with one or more hormone therapy drugs; and (b) administering a therapeutically effective amount of the one or more hormone therapy drugs to the selected subject based on expression of ER, Ki-67, and PR as described herein. In some embodiments, the selection of the subject for the treatment with the one or more hormone therapy drugs includes (i) obtaining a biological sample from the subject having the cancer; (ii) assessing the expression of the ER, Ki-67, and PR biomarkers as described herein in the obtained biological sample; and (iii) selecting the subject candidate for treatment with the one or more hormone therapy drugs if the obtained biological sample is determined to be an ER+, Ki-67+, PR+ dominant case.

Methods of Stratifying and/or Classifying Patients

The present disclosure also provides methods of stratifying patients or patient populations into likely to have a recurrence versus those not likely to have a recurrence. Indeed, Applicant has discovered that the triplex IHC assays of the present disclosure may be used to predict the recurrence of ER-positive breast cancers. Applicant has also discovered that the triplex IHC assays of the present disclosure may be used to predict the recurrence of ER-positive breast cancers without the need of conducting any breast cancer gene expression tests. As noted in the example herein, ER+, Ki-67+, PR+ dominant patients had significantly better clinical outcomes than ER+, Ki-67+ dominant patients with hormone therapy among ER-positive and HER2-negative breast cancer populations.

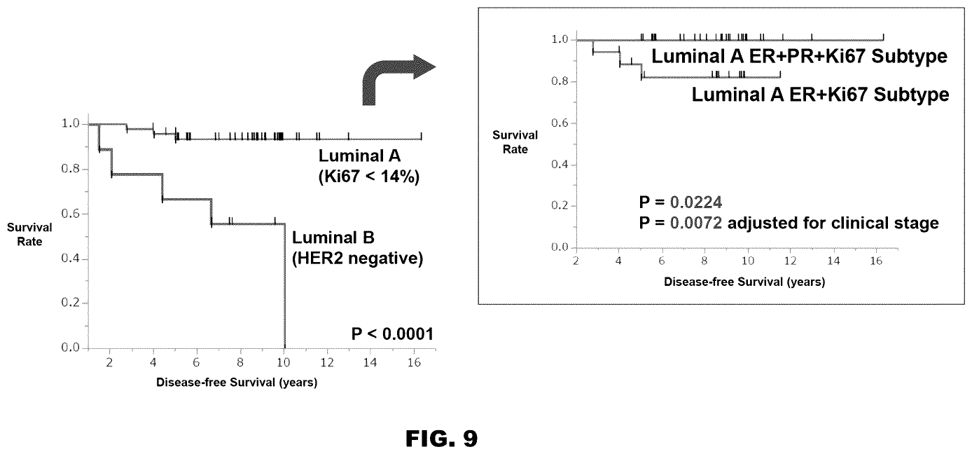

Additionally, Applicant has discovered that Luminal A subtype patients may be reclassified to ER+, Ki-67+, PR+ and ER+, Ki-67+ subtypes. Applicant has shown that ER+, Ki-67+, PR+ dominant patients had significantly better clinical outcomes than ER+, Ki-67+ dominant patients (see, FIG. 9).

### KITS

The present disclosure also provides for kits including antibodies and detection reagents suitable for staining a sample in a multiplex or simplex immunoenzymatic assay. In some embodiments, the kit includes (i) a set primary antibodies, and (ii) optionally detection agents for performing an immunoenzymatic assay, wherein the set of primary antibodies includes anti-ER antibodies, anti-Ki-67 antibodies, and anti-PR antibodies. In some embodiments, the anti-ER, anti-Ki-67, and anti-PR antibodies are monoclonal antibodies. In some embodiments, the anti-ER, anti-Ki-67, and anti-PR antibodies are mouse monoclonal antibodies. In some embodiments, the anti-ER, anti-Ki-67, and anti-PR antibodies are rabbit monoclonal antibodies. In some embodiments, the human PR biomarker-specific reagent is Clone 1E2. In some embodiments, the human ER biomarker-specific reagent is SP1. In some embodiments, the human Ki-67 biomarker-specific reagent is Clone 30-9. In some embodiments, the set of primary antibodies comprises the Clone 1E2, the SP1 clone, and Clone 30-9. In other embodiments, the set of primary antibodies consists essentially of the Clone 1E2, the SP1 clone, and Clone 30-9. In some embodiments, the set of primary antibodies consists of the Clone 1E2, the SP1 clone, and Clone 30-9.

In some embodiments, the kit further includes secondary antibodies specific to the anti-ER antibody, the anti-Ki-67 antibody, and the anti-PR antibody. In some embodiments, the secondary antibodies are conjugated to either a peroxidase enzyme or an alkaline phosphatase enzyme.

In some embodiments, the kit further includes first, second, and third brightfield detectable moieties. In some embodiments, the first, second, and third brightfield detectable moieties are selected from TAMRA, Dabsyl, Dabcyl, Cy3, CyB, Cy3.5, Cy5, Cy5.5, Cy7, rhodamine 800 and fluorescein. In some embodiments, the first, second, and third brightfield detectable moieties include a conjugate comprising at least two chromogens. In some embodiments, the brightfield detectable moieties are selected from Discovery Yellow, Discovery Teal, and Discovery Purple, each available from Ventana Medical Systems, Inc. (Tucson, AZ).

### Example—Co-Expression of Estrogen Receptor (ER), Progesterone Receptor (PR), and Ki-67 in a Single Breast Cancer Cell Indicates a Favorable Prognosis in ER-Positive Breast Cancer

### Background

ER-positive breast cancer is biologically and clinically divided into two subtypes: luminal-A and luminal-B. This classification is mainly based on the status of cellular proliferation. At least two different pathways drive cellular proliferation in ER-positive breast cancer. One is a classical pathway where ER binds to estrogen responsive element (ERE), which leads to the expression of downstream molecules, including PR. The other is a non-classical pathway where a complex of ER and relevant factors bind to different sites than ERE. Growth factor signaling has been suggested to potentiate a non-classical pathway. It was hypothesized that examining PR status in ER-positive proliferating cells could tell which pathway is more dominant in ER-positive breast cancer.

### Methods

To test the hypothesis, a newly developed triplex immunohistochemistry (IHC) assay was used that detects three molecules simultaneously under bright-field microscopy. Postmenopausal patients who were treated with neoadjuvant endocrine therapy with aromatase inhibitors from January 2007 to September 2016 at Saitama Cancer Center were included in this study. ER, PR, and Ki-67 expressions were assessed in a single slide using the triplex IHC assay with anti-ER antibody (clone SP1), anti-PR antibody (clone 1E2), and anti-Ki-67 antibody (clone 30-9). ER, PR and Ki-67 expression was assessed in a single cell nucleus of cancer cells (567 to 4871 cells) from multiple areas in each case. An ER-positive proliferating cell was defined as an ER-positive and Ki-67-positive cell. PR status in ER-positive proliferating cells was assessed. When PR was expressed in more than 50% of ER-positive proliferating cells in a clinical case, the tumor was categorized as the PR-positive group. Luminal A and luminal B breast cancers were defined based on the pre-treatment Ki-67 labeling index with a cut-off of 14%. Statistical analyses included the Mann-Whitney test, the log-rank test, and the Cox proportional hazard model.

### Results

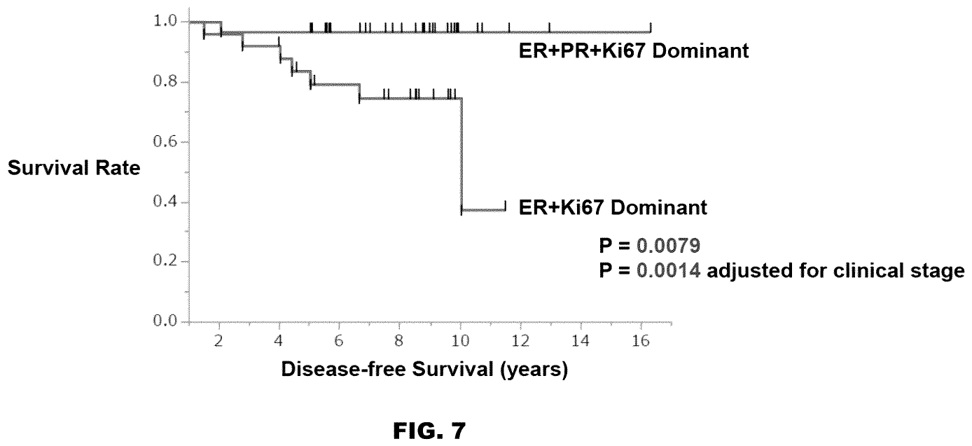

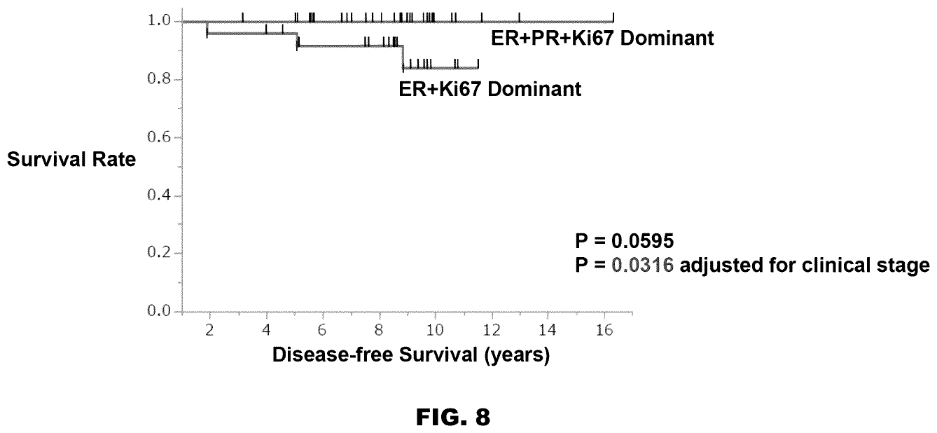

Pre-treatment tissues from 55 patients were evaluated (see Table, below). The median age was 62 (range: 54 to 80) years. The patients were grouped into PR-positive and PR-negative groups. The two groups had no differences in age and pre-treatment T and N stages. The median pre-treatment Ki-67 labeling index was 5.9% in the PR-positive group and 9.9% in the PR-negative group, which showed a statistically significant difference (P=0.01). Clinical response to neoadjuvant endocrine therapy was compared, and no difference was observed. The median post-treatment Ki-67 labeling index was 3.6% in the PR-positive group and 13.1% in the PR-negative group with a significant difference (P=0.035). The survival was compared between the two groups. The PR-positive group showed a significantly more favorable disease-free survival (DFS) than the PR-negative group (P=0.0079). To adjust for the background differences, a multivariate analysis showed that the PR-positive group had a significantly better DFS than the PR-negative group independent of clinical stage, Ki-67 labeling index, and PR status (P=0.042) (see, e.g., FIG. 7). Breast cancer-specific survival (BCSS) was also better in the PR-positive group than in the PR-negative group after adjusting for clinical stage, Ki-67 labeling index, and PR status (P=0.043) (see, e.g., FIG. 8). Interestingly, among patients with luminal A tumors, those in the PR-positive group showed a better DFS than those in the PR-negative group (P=0.022) (see, e.g., FIG. 9).

| Subtype | Biomarker Status |
|---|---|
| Luminal A | ER+, PR+/−, HER2−, Ki-67 < 14% |
| Luminal B - HER2− | ER+, PR+/−, HER2−, Ki-67 > 14% |
| Luminal B - HER2+ | ER+, PR+/−, HER2+ |
| HER2+ | ER−, PR−, HER2+ |
| Triple Negative | ER−, PR−, HER2− |

### CONCLUSION

PR status in ER-positive proliferating cells was an independent prognostic factor in DFS and BCSS and divided patients with luminal A tumors further into two prognostic groups.

### Additional Embodiments

Another aspect of the present disclosure is an immunohistochemical method for assessing expression of the PR, ER, and Ki-67 biomarkers in a sample, the method comprising: contacting the sample with a human PR biomarker-specific reagent under conditions that permit specific binding of the PR biomarker-specific reagent to the sample; contacting the sample with a first set of detection reagents that interact with the human PR biomarker-specific reagent to facilitate deposition of a first brightfield detectable moiety on the sample; contacting the sample with a human ER biomarker-specific reagent under conditions that permit specific binding of the ER biomarker-specific reagent to the sample; contacting the sample with a second set of detection reagents that interact with the human ER biomarker-specific reagent to facilitate deposition of a second brightfield detectable moiety on the sample; contacting the sample with a human Ki-67 biomarker-specific reagent under conditions that permit specific binding of the Ki-67 biomarker-specific reagent to the sample; contacting the sample with a third set of detection reagents that interact with the human Ki-67 biomarker-specific reagent to facilitate deposition of a third brightfield detectable moiety on the sample; and identifying cell nuclei within the sample expressing each of the PR, ER, and Ki-67 biomarkers based on a coexpression of signals from each of the deposited first, second, and third brightfield detectable moieties.

Another aspect of the present disclosure is an immunohistochemical method for assessing expression of the PR and ER biomarkers and a biomarker of cellular proliferation (e.g., Ki-67) in a sample, the method comprising: contacting the sample with a human PR biomarker-specific reagent under conditions that permit specific binding of the PR biomarker-specific reagent to the sample; contacting the sample with a first set of detection reagents that interact with the human PR biomarker-specific reagent to facilitate deposition of a first brightfield detectable moiety on the sample; contacting the sample with a human ER biomarker-specific reagent under conditions that permit specific binding of the ER biomarker-specific reagent to the sample; contacting the sample with a second set of detection reagents that interact with the human ER biomarker-specific reagent to facilitate deposition of a second brightfield detectable moiety on the sample; contacting the sample with a human cell proliferation biomarker-specific reagent under conditions that permit specific binding of the cell proliferation biomarker-specific reagent to the sample; contacting the sample with a third set of detection reagents that interact with the human cell proliferation biomarker-specific reagent to facilitate deposition of a third brightfield detectable moiety on the sample; and identifying cell nuclei within the sample expressing each of the PR, ER, and cell proliferation biomarkers based on a coexpression or colocalization of signals from each of the deposited first, second, and third brightfield detectable moieties.

Another aspect of the present disclosure comprises staining a first of the ER, BR, and Ki-67 biomarkers in a sample with a yellow stain or a yellow-like stain; staining a second of the ER, PR, and Ki-67 biomarkers in the sample with a teal or teal-like stain; and staining a third of the ER, PR, and Ki-67 biomarkers in a sample with a purple or purple-like stain. In some embodiments, the sample is assessed to determinate a colocalization of signals from the yellow stain (or yellow-like stain), the teal stain (or teal-like stain), and the purple stain (or purple-like stain). In some embodiments, the sample is a breast tumor sample. In some embodiments, the sample is a sample previously diagnosed as Luminal A breast cancer.

Another aspect of the disclosure is an affinity histochemical or affinity cytochemical method for assessing expression of the PR, ER, and Ki-67 biomarkers in a sample, the method comprising: (a) contacting the sample with a human ER biomarker-specific reagent under conditions that permit specific binding of the ER biomarker-specific reagent to the sample; (b) contacting the sample with a first set of detection reagents that interact with the human ER biomarker-specific reagent to facilitate deposition of a first brightfield detectable moiety on the sample; (c) contacting the sample with a human PR biomarker-specific reagent under conditions that permit specific binding of the PR biomarker-specific reagent to the sample; (d) contacting the sample with a second set of detection reagents that interact with the human PR biomarker-specific reagent to facilitate deposition of a second brightfield detectable moiety on the sample; (e)contacting the sample with a human Ki-67 biomarker-specific reagent under conditions that permit specific binding of the Ki-67 biomarker-specific reagent to the sample; (f) contacting the sample with a third set of detection reagents that interact with the human Ki-67 biomarker-specific reagent to facilitate deposition of a third brightfield detectable moiety on the sample; and (g) identifying cell nuclei within the sample expressing each of the PR, ER, and Ki-67 biomarkers based on a colocalization of signals from each of the deposited first, second, and third brightfield dyes.

Another aspect of the disclosure is an affinity histochemical or affinity cytochemical method for assessing expression of the PR, ER, and Ki-67 biomarkers in a sample, the method comprising: (a)contacting the sample with a human ER biomarker-specific reagent under conditions that permit specific binding of the ER biomarker-specific reagent to the sample; (b) contacting the sample with a first set of detection reagents that interact with the human ER biomarker-specific reagent to facilitate deposition of a first brightfield detectable moiety on the sample; (c) contacting the sample with a human Ki-67 biomarker-specific reagent under conditions that permit specific binding of the Ki-67 biomarker-specific reagent to the sample; (d) contacting the sample with a second set of detection reagents that interact with the human Ki-67 biomarker-specific reagent to facilitate deposition of a second brightfield detectable moiety on the sample; (e) contacting the sample with a human PR biomarker-specific reagent under conditions that permit specific binding of the PR biomarker-specific reagent to the sample; (f) contacting the sample with a third set of detection reagents that interact with the human PR biomarker-specific reagent to facilitate deposition of a third brightfield detectable moiety on the sample; and (g) identifying cell nuclei within the sample expressing each of the PR, ER, and Ki-67 biomarkers based on a colocalization of signals from each of the deposited first, second, and third brightfield dyes.

Another aspect of the disclosure is an affinity histochemical or affinity cytochemical method for assessing expression of the PR, ER, and Ki-67 biomarkers in a sample, the method comprising: (a) contacting the sample with a human Ki-67 biomarker-specific reagent under conditions that permit specific binding of the Ki-67 biomarker-specific reagent to the sample; (b) contacting the sample with a first set of detection reagents that interact with the human Ki-67 biomarker-specific reagent to facilitate deposition of a first brightfield detectable moiety on the sample; (c) contacting the sample with a human ER biomarker-specific reagent under conditions that permit specific binding of the ER biomarker-specific reagent to the sample; (d) contacting the sample with a second set of detection reagents that interact with the human ER biomarker-specific reagent to facilitate deposition of a second brightfield detectable moiety on the sample; (e) contacting the sample with a human PR biomarker-specific reagent under conditions that permit specific binding of the PR biomarker-specific reagent to the sample; (f) contacting the sample with a third set of detection reagents that interact with the human PR biomarker-specific reagent to facilitate deposition of a third brightfield detectable moiety on the sample; and (g) identifying cell nuclei within the sample expressing each of the PR, ER, and Ki-67 biomarkers based on a colocalization of signals from each of the deposited first, second, and third brightfield dyes.

Another aspect of the disclosure is an affinity histochemical or affinity cytochemical method for assessing expression of the PR, ER, and Ki-67 biomarkers in a sample, the method comprising: contacting the sample with a human Ki-67 biomarker-specific reagent, a human PR biomarker-specific reagent, and a human ER biomarker-specific reagent under conditions that permit specific binding of the human Ki-67 biomarker-specific reagent, the human PR biomarker-specific reagent, and the human ER biomarker-specific reagent to the sample; sequentially contacting the sample with first, second, and third detection reagents that interact with the human Ki-67 biomarker specific reagent, the human PR biomarker-specific reagent, and the human ER biomarker-specific reagents to facilitate deposition of a first brightfield detectable moiety, a second brightfield detectable moiety, and a third brightfield detectable moiety on the sample; and identifying cell nuclei within the sample expressing each of the Ki-67, PR, and ER biomarkers based on a colocalization of signals from each of the deposited first, second, and third detectable moieties. In some embodiments, the contacting of the sample with the human Ki-67 biomarker-specific reagent, the human PR biomarker-specific reagent, and the human ER biomarker-specific reagent may be conducted in any sequential order. In some embodiments, the contacting of the sample with the human Ki-67 biomarker-specific reagent, the human PR biomarker-specific reagent, and the human ER biomarker-specific reagent may be conducted simultaneously.

Another aspect of the present disclosure is a kit comprising: (i) a human Ki-67 biomarker-specific reagent, (ii) a human PR biomarker-specific reagent, and (iii) a human ER biomarker-specific reagent. In some embodiments, the kit further comprises first, second, and third detection reagents. In some embodiments, the first, second, and third detection reagents each comprise a different detectable moiety. In some embodiments, the different detectable moiety is a chromogen. In some embodiments, the different detectable moiety is selected from the group consisting of TAMRA, Dabsyl, Dabcyl, Cy3, CyB, Cy3.5, Cy5, Cy5.5, Cy7, rhodamine 800 and fluorescein. In some embodiments, the human Ki-67 biomarker-specific reagent is a monoclonal antibody. In some embodiments, the monoclonal antibody is selected from the group consisting of MIB-1, MIB-2, MIB-5, MIB-7, MIB-21, MIB-24, and clone 30-9. In some embodiments, the human ER biomarker-specific reagent is a monoclonal antibody. In some embodiments, the monoclonal antibody is 1D5. In some embodiments, the human PR biomarker-specific reagent is a monoclonal antibody. In some embodiments, the monoclonal antibody is a 1E2 clone.

Another aspect of the present disclosure is a method of selecting a patient with a breast tumor to receive a hormone therapy, the method comprising: (a) contacting the sample with a human PR biomarker-specific reagent under conditions that permit specific binding of the PR biomarker-specific reagent to the sample; (b) contacting the sample with a first set of detection reagents that interact with the human PR biomarker-specific reagent to facilitate deposition of a first brightfield detectable moiety on the sample; (c) contacting the sample with a human ER biomarker-specific reagent under conditions that permit specific binding of the ER biomarker-specific reagent to the sample; (d) contacting the sample with a second set of detection reagents that interact with the human ER biomarker-specific reagent to facilitate deposition of a second brightfield detectable moiety on the sample; (e) contacting the sample with a human Ki-67 biomarker-specific reagent under conditions that permit specific binding of the Ki-67 biomarker-specific reagent to the sample; (f) contacting the sample with a third set of detection reagents that interact with the human Ki-67 biomarker-specific reagent to facilitate deposition of a third brightfield detectable moiety on the sample; (g) identifying cell nuclei within the sample expressing each of the PR, ER, and Ki-67 biomarkers based on a colocalization of signals from each of the deposited first, second, and third brightfield detectable moieties; and (h) determining a number of proliferating tumor nuclei that are both ER+ and PR+ within the histochemically stained sample; wherein the patient is selected to receive the hormone therapy if a ratio of the determined number of proliferating tumor nuclei that are both ER+ and PR+ to a total number of ER+proliferating tumor nuclei is greater or equal to a predetermined cutoff value; wherein the predetermined cutoff value is between about 0.4 to about 0.6.

Another aspect of the present disclosure is a method of selecting a patient with a breast tumor to receive a hormone therapy, the method comprising: (a) contacting the sample with a human PR biomarker-specific reagent under conditions that permit specific binding of the PR biomarker-specific reagent to the sample; (b) contacting the sample with a first set of detection reagents that interact with the human PR biomarker-specific reagent to facilitate deposition of a first brightfield detectable moiety on the sample; (c) contacting the sample with a human ER biomarker-specific reagent under conditions that permit specific binding of the ER biomarker-specific reagent to the sample; (d) contacting the sample with a second set of detection reagents that interact with the human ER biomarker-specific reagent to facilitate deposition of a second brightfield detectable moiety on the sample; (e) contacting the sample with a human Ki-67 biomarker-specific reagent under conditions that permit specific binding of the Ki-67 biomarker-specific reagent to the sample; (f) contacting the sample with a third set of detection reagents that interact with the human Ki-67 biomarker-specific reagent to facilitate deposition of a third brightfield detectable moiety on the sample; (g) identifying cell nuclei within the sample expressing each of the PR, ER, and Ki-67 biomarkers based on a colocalization of signals from each of the deposited first, second, and third brightfield detectable moieties; and (h) calculating a ratio of a number of cells or cell nuclei that stain positive for all three of ER, Ki-67, and PR to the total number of cells that are both (i) ER+,Ki-67+, and PR+, and (ii) ER+, Ki-67+, and PR−; wherein the patient is selected to receive the hormone therapy if the calculated ratio is greater than or equal to a predetermined cutoff value; wherein the predetermined cutoff value is between about 0.4 to about 0.6.

Another aspect of the present disclosure is a method of selecting a patient with a breast tumor to receive a hormone therapy, the method comprising: (a) contacting the sample with a human PR biomarker-specific reagent under conditions that permit specific binding of the PR biomarker-specific reagent to the sample; (b) contacting the sample with a first set of detection reagents that interact with the human PR biomarker-specific reagent to facilitate deposition of a first brightfield detectable moiety on the sample; (c) contacting the sample with a human ER biomarker-specific reagent under conditions that permit specific binding of the ER biomarker-specific reagent to the sample; (d) contacting the sample with a second set of detection reagents that interact with the human ER biomarker-specific reagent to facilitate deposition of a second brightfield detectable moiety on the sample; (e) contacting the sample with a human Ki-67 biomarker-specific reagent under conditions that permit specific binding of the Ki-67 biomarker-specific reagent to the sample; (f) contacting the sample with a third set of detection reagents that interact with the human Ki-67 biomarker-specific reagent to facilitate deposition of a third brightfield detectable moiety on the sample; (g) identifying cell nuclei within the sample expressing each of the PR, ER, and Ki-67 biomarkers based on a colocalization of signals from each of the deposited first, second, and third brightfield detectable moieties; and (h) calculating a ratio of a number of cells or cell nuclei that stain positive for all three of ER, Ki-67, and PR to the total number of cells that are (i) ER+, Ki-67+, and PR+, and (ii) ER+, Ki-67+, and PR−; wherein the patient is selected to receive the hormone therapy if the calculated ratio is less than a predetermined cutoff value; wherein the predetermined cutoff value is between about 0.4 to about 0.6.

Another aspect of the present disclosure is a method of classifying a patient with breast cancer as either ER+, Ki-67+, PR+ dominant or ER+, Ki-67+ dominant, the method comprising: (a) contacting the sample with a human PR biomarker-specific reagent under conditions that permit specific binding of the PR biomarker-specific reagent to the sample; (b) contacting the sample with a first set of detection reagents that interact with the human PR biomarker-specific reagent to facilitate deposition of a first brightfield detectable moiety on the sample; (c) contacting the sample with a human ER biomarker-specific reagent under conditions that permit specific binding of the ER biomarker-specific reagent to the sample; (d) contacting the sample with a second set of detection reagents that interact with the human ER biomarker-specific reagent to facilitate deposition of a second brightfield detectable moiety on the sample; (e) contacting the sample with a human Ki-67 biomarker-specific reagent under conditions that permit specific binding of the Ki-67 biomarker-specific reagent to the sample; (f) contacting the sample with a third set of detection reagents that interact with the human Ki-67 biomarker-specific reagent to facilitate deposition of a third brightfield detectable moiety on the sample; (g) identifying cell nuclei within the sample expressing each of the PR, ER, and Ki-67 biomarkers based on a colocalization of signals from each of the deposited first, second, and third brightfield detectable moieties; and calculating a ratio of a number of cells or cell nuclei that stain positive for all three of ER, Ki-67, and PR to the total number of cells that are (i) ER+, Ki-67+, and PR+, and (ii) ER+, Ki-67+, and PR−; wherein the patient is classified as ER+, Ki-67+, PR+ dominant if the calculated ratio is greater than or equal to a predetermined threshold; or the patient is classified as ER+, Ki-67+ dominant if the calculated ratio is less than the predetermined threshold.

Another aspect of the present disclosure is a method of classifying a patient with breast cancer as a likely responder or a likely non-responder to hormone treatment, the method comprising: (a) contacting the sample with a human PR biomarker-specific reagent under conditions that permit specific binding of the PR biomarker-specific reagent to the sample; (b) contacting the sample with a first set of detection reagents that interact with the human PR biomarker-specific reagent to facilitate deposition of a first brightfield detectable moiety on the sample; (c) contacting the sample with a human ER biomarker-specific reagent under conditions that permit specific binding of the ER biomarker-specific reagent to the sample; (d) contacting the sample with a second set of detection reagents that interact with the human ER biomarker-specific reagent to facilitate deposition of a second brightfield detectable moiety on the sample; (e) contacting the sample with a human Ki-67 biomarker-specific reagent under conditions that permit specific binding of the Ki-67 biomarker-specific reagent to the sample; (f) contacting the sample with a third set of detection reagents that interact with the human Ki-67 biomarker-specific reagent to facilitate deposition of a third brightfield detectable moiety on the sample; (g) identifying cell nuclei within the sample expressing each of the PR, ER, and Ki-67 biomarkers based on a colocalization of signals from each of the deposited first, second, and third brightfield detectable moieties; and calculating a ratio of a number of cells or cell nuclei that stain positive for all three of ER, Ki-67, and PR to the total number of cells that are (i) ER+, Ki-67+, and PR+, and (ii) ER+, Ki-67+, and PR−; wherein the patient is classified as likely responder if the calculated ratio is greater than or equal to a predetermined threshold; or the patient is classified as a likely non-responder if the calculated ratio is less than the predetermined threshold.

All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications, and publications to provide yet further embodiments.

Although the present disclosure has been described with reference to a number of illustrative embodiments, it should be understood that numerous other modifications and embodiments can be devised by those skilled in the art that will fall within the spirit and scope of the principles of this disclosure. More particularly, reasonable variations and modifications are possible in the component parts and/or arrangements of the subject combination arrangement within the scope of the foregoing disclosure, the drawings, and the appended claims without departing from the spirit of the disclosure. In addition to variations and modifications in the component parts and/or arrangements, alternative uses will also be apparent to those skilled in the art.

1. An affinity histochemical or affinity cytochemical method for assessing expression of the PR, ER, and Ki-67 biomarkers in a sample, the method comprising:
(a) contacting the sample with a human PR biomarker-specific reagent under conditions that permit specific binding of the PR biomarker-specific reagent to the sample;
(b) contacting the sample with a first set of detection reagents that interact with the human PR biomarker-specific reagent to facilitate deposition of a first brightfield detectable moiety on the sample;
(c) contacting the sample with a human ER biomarker-specific reagent under conditions that permit specific binding of the ER biomarker-specific reagent to the sample;
(d) contacting the sample with a second set of detection reagents that interact with the human ER biomarker-specific reagent to facilitate deposition of a second brightfield detectable moiety on the sample;
(e) contacting the sample with a human Ki-67 biomarker-specific reagent under conditions that permit specific binding of the Ki-67 biomarker-specific reagent to the sample;
(f) contacting the sample with a third set of detection reagents that interact with the human Ki-67 biomarker-specific reagent to facilitate deposition of a third brightfield detectable moiety on the sample; and
(g) identifying cell nuclei within the sample expressing each of the PR, ER, and Ki-67 biomarkers based on a colocalization of signals from each of the deposited first, second, and third brightfield detectable moieties.

2. The method of claim 1, wherein the human PR biomarker-specific reagent is an anti-PR monoclonal antibody, wherein the human ER biomarker-specific reagent is an anti-ER monoclonal antibody, and wherein the human Ki-67 biomarker-specific reagent is an anti-Ki-67 monoclonal antibody.

3. The method of claim 1, wherein the first set of detection reagents include: (i) a first secondary antibody specific to the human PR biomarker-specific reagent; and (ii) a conjugate including the first brightfield detectable moiety.

4. The method of claim 3, wherein the first secondary antibody specific to the human PR biomarker-specific reagent includes a first enzyme.

5. The method of claim 1, wherein the second set of detection reagents include: (i) a second secondary antibody specific to the human ER biomarker-specific reagent; and (ii) a conjugate including the second brightfield detectable moiety.

6. The method of claim 5, wherein the second secondary antibody specific to the human ER biomarker-specific reagent includes a first enzyme.

7. The method of claim 1, wherein the third set of detection reagents include: (i) a third secondary antibody specific to the human Ki-67 biomarker-specific reagent; and (ii) a conjugate including the third brightfield detectable moiety.

8. The method of claim 7, wherein the third secondary antibody specific to the human Ki-67 biomarker-specific reagent includes a first enzyme.

9. The method of claim 1, wherein the first, second, and third brightfield detectable moieties are each independently selected from the group consisting of TAMRA, Dabsyl, Dabcyl, Cy3, CyB, Cy3.5, Cy5, Cy5.5, Cy7, rhodamine 800 and fluorescein.

10. The method of claim 1, wherein the sample is a breast tissue sample from a subject diagnosed with breast cancer.

11. The method of claim 10, wherein the breast cancer is Luminal A breast cancer.

12. The method of claim 1, wherein a first inactivation composition is applied to the sample prior to the contacting of the sample with the human ER biomarker-specific reagent, and wherein a second inactivation composition is applied to the sample prior to the contacting of the sample with the human Ki-67 biomarker-specific reagent.

13. A method of selecting a patient with a breast tumor to receive a hormone therapy, the method comprising:
(a) affinity histochemically staining a sample derived from the breast tumor with a human PR biomarker-specific reagent under conditions that permit specific binding of the PR biomarker-specific reagent to the sample;
(b) affinity histochemically staining the sample derived from the breast tumor with a human ER biomarker-specific reagent under conditions that permit specific binding of the ER biomarker-specific reagent to the sample;
(c) affinity histochemically staining the sample derived from the breast tumor with a human Ki-67 biomarker-specific reagent under conditions that permit specific binding of the Ki-67 biomarker-specific reagent to the sample; and
(d) determining a number of proliferating tumor nuclei that are both ER+ and PR+ within the histochemically stained sample;
wherein the patient is selected to receive the hormone therapy if a ratio of the determined number of proliferating tumor nuclei that are both ER+ and PR+ to a total number of ER+proliferating tumor nuclei is greater or equal to a predetermined cutoff value.

14. The method of claim 13, wherein the predetermined cutoff value is between about 0.4 to about 0.6.

15. The method of claim 13, wherein the predetermined cutoff value is between about 0.45 to about 0.55.

16. The method of claim 13, wherein the predetermined cutoff value is about 0.5.

17. The method of claim 13, wherein the hormone therapy is a selective estrogen receptor modulator, a selective estrogen receptor degrader, or an aromatase inhibitor.

18. The method of claim 13, wherein the patient was previously diagnosed with Luminal A breast cancer.

19. The method of claim 13, wherein the patient was previously treated with endocrine therapy, or wherein the patient was previously treated with adjuvant chemotherapy.

20. The method of claim 13, wherein the affinity histochemical staining of the sample with the human PR biomarker-specific reagent comprises (i) contacting the sample with a human PR biomarker-specific reagent under conditions that permit specific binding of the PR biomarker-specific reagent to the sample; and (ii) contacting the sample with a first set of detection reagents that interact with the human PR biomarker-specific reagent to facilitate deposition of a first brightfield detectable moiety on the sample; wherein the affinity histochemical staining of the sample with the human ER biomarker-specific reagent comprises (i) contacting the sample with a human ER biomarker-specific reagent under conditions that permit specific binding of the ER biomarker-specific reagent to the sample; and (ii) contacting the sample with a second set of detection reagents that interact with the human ER biomarker-specific reagent to facilitate deposition of a second brightfield detectable moiety on the sample; and wherein the affinity histochemical staining of the sample with the human Ki-67 biomarker-specific reagent comprises (i) contacting the sample with a human Ki-67 biomarker-specific reagent under conditions that permit specific binding of the Ki-67 biomarker-specific reagent to the sample; and (ii) contacting the sample with a third set of detection reagents that interact with the human Ki-67 biomarker-specific reagent to facilitate deposition of a third brightfield detectable moiety on the sample.
